(19) United States
(12) Patent Application Publication
(10) Pub. No.: US 2017/0044100 A1
(43) Pub. Date: Feb. 16, 2017

(54) INHIBITORS OF DRUG-RESISTANT MYCOBACTERIUM TUBERCULOSIS

(71) Applicants: THE JOHNS HOPKINS UNIVERSITY, Baltimore, MD (US); UNIVERSITY OF ILLINOIS AT CHICAGO, Chicago, IL (US)

(72) Inventors: William R. Bishai, Towson, MD (US); Shichun Lun, Ellicott City, MD (US); Haidan Guo, Ellicott City, MD (US); Alan Kozikowski, Chicago, IL (US); Oluseye Onajole, Forest Park, IL (US); Jozef Stec, Tinley Park, IL (US)

(21) Appl. No.: 15/305,725

(22) PCT Filed: Apr. 22, 2015
(86) PCT No.: PCT/US2015/027053

§ 371 Date: Oct. 21, 2016

Related U.S. Application Data
(60) Provisional application No. 61/982,685, filed on Apr. 22, 2014.

(51) Int. Cl. C07D 209/42; C07D 403/12; C07D 307/85; C07D 235/24; C07D 487/04; C07D 277/68; A61K 31/404; A61K 31/4439; A61K 31/454; A61K 31/55; A61K 31/343; A61K 31/407; A61K 31/428; A61K 31/4409; A61K 31/496; C07D 401/12

(52) U.S. Cl. CPC C07D 209/42; C07D 401/12; C07D 403/12; C07D 307/85; C07D 235/24; C07D 487/04; C07D 277/68; A61K 31/404; A61K 31/4439; A61K 31/454; A61K 31/55; A61K 31/343; A61K 31/407; A61K 31/428; A61K 31/4409; A61K 31/496

(57) ABSTRACT

The present invention provides novel indoleamide compounds for treating tuberculosis, including drug-resistant M-tuberculosis, compositions comprising the indoleamides and methods of using the indoleamides in conjunction with other biologically active agents for the treatment of tuberculosis in a subject in need thereof.

### REFERENCE TO RELATED APPLICATIONS

This application claims the benefit of U.S. Provisional Patent Application No. 61/982,685, filed on Apr. 22, 2014, which is hereby incorporated by reference for all purposes as if fully set forth herein.

### TECHNICAL FIELD

This invention relates to novel indoleamide compounds for treating tuberculosis, including drug-resistant M-tuberculosis, compositions comprising the indoleamides and methods of using the indoleamides.

### BACKGROUND OF THE INVENTION

Tuberculosis (TB) is a human infectious disease responsible for significant worldwide morbidity and mortality, accountable for an estimated 8.7 million incident cases and 1.4 million deaths in 2011¹. Although effective therapy exists for TB caused by drug-susceptible Mycobacterium tuberculosis, this therapy requires daily administration of multiple drugs for a minimum of 6 months. Strict adherence to treatment is necessary for successful outcome. However, the intensity and duration of effective therapy challenge patient compliance and thus contribute to treatment failures, leading to increased disease, continued M. tuberculosis transmission and ultimately selection of drug-resistant organisms. The development of drug resistance is especially alarming, as transmission of drug-resistant bacilli can lead to primary infections refractory to standard TB therapy. In 2011, the World Health Organization (WHO) reported that 3.7% of new TB cases were due to infection with multidrug-resistant (MDR) M. tuberculosis¹. The tragic development of MDR- and extensively drug-resistant-(XDR-) TB has kindled a worldwide push for the development of new therapy options for this disease, and new drugs are desperately needed to enable effective worldwide TB control.

The current WHO-endorsed standard regimen for the treatment of drug-susceptible TB consists of daily rifampin, isoniazid, pyrazinamide and ethambutol for two months, followed by four months of daily isoniazid and rifampin. This first-line regimen, referred to as the “short course” (as previous treatment regimens ranged from 18-24 months in duration), utilizes some of the oldest antibiotics in modern medicine, with isoniazid and pyrazinamide developed in the 1950s and ethambutol and rifampin developed in the 1960s. That the most recent first-line anti-TB drugs are over 50 years old illustrates the paucity of drug development advances in this field.

In December 2012, the United States Food and Drug Administration (FDA) granted accelerated approval of bedaquiline, a diarylquinoline antimycobacterial drug, for the treatment of MDR-TB (infection with M. tuberculosis resistant to rifampin and isoniazid), including XDR-TB (resistance to rifampin, isoniazid, a quinolone and one of the injectable drugs: kanamycin, amikacin or capreomycin), when no other treatment options exist². The FDA approval of bedaquiline is a landmark event in TB chemotherapy, representing the introduction of a new drug class and being the first new TB drug approved in half a century. However, the nature of the approval, being only permitted for use when other treatment options are exhausted, indicates that bedaquiline will be added to otherwise failing drug regimens, and as such it can be anticipated that microbial resistance to this new compound will eventually emerge. Thus, it is imperative that TB drug development efforts continue to push forward.

### SUMMARY OF THE INVENTION

We have designed a series of indoleamides with potent activity against both drug-susceptible and drug-resistant strains of M. tuberculosis by targeting the mycolic acid transporter MmpL3. We identify a single mutation in mmpL3 which confers high resistance to the indoleamide class while remaining susceptible to currently used first- and second-line tuberculosis drugs, signifying a lack of cross-resistance. Importantly, an indoleamide derivative exhibits dose-dependent anti-mycobacterial activity when orally administered to M. tuberculosis-infected mice. The bioavailability of the indoleamides, combined with their ability to kill tubercle bacilli, indicates great potential for translational developments of this structure class for the treatment of drug-resistant tuberculosis.

In its principle aspect, this invention is a compound of formula I:

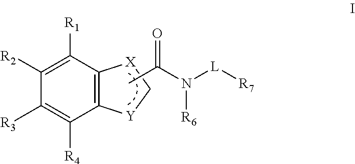

wherein
R₁, R₂, R₃ and R₄ are independently selected from H, alkyl, haloalkyl, alkoxy, halo and amino; X is CH, N or S; Y is O or NR₅; L is absent or C₁-C₄ alkyl; R₆ is H or alkyl; R₇ is C₃-C₁₀ cycloalkyl, C₅-C₈ heterocyclyl, C₆ aryl, C₅-C₆ heteroaryl or alkyl, or R₆ and R₇ together form a C₅-C₈ heterocyclyl; and R₅ is H or alkyl, or a pharmaceutically acceptable salt, solvate or stereoisomer thereof.

### BRIEF DESCRIPTION OF THE DRAWINGS

FIG. 1 shows the serum inhibition titration result for compound 12. nNH=number of hydrogen bond donors; nON=number of hydrogen bond acceptors; MW=Molecular Weight; TPSA=Topological polar surface area; nRot. bond=number of rotatable bonds calculated using the molinspiration online service (www.molinspiration.com); ClogD was calculated using the ACD/lab Percepta software; BALB/c mice were orally gavaged with two doses (100 and 300 mg/kg) of compound 12, with blood collected at different time points and serum separated 60 min later. Growth inhibition of serially diluted serum on H37Rv was determined using the Alamar Blue assay; Vehicle, 0.5% CMC (carboxylmethyl cellulose); INH, isoniazid at 10 mg/kg (positive control).

FIGS. 2A-2C show that indoleamide compounds are active in vitro against Mycobacterium tuberculosis. (a) Structure of compound 1, the initial hit indoleamide. (b) Structures of compounds 11 and 12, derivatives of compound 3. (c) In vitro kill curve of M. tuberculosis exposed to 4× and 16× MIC of the indoleamide derivative compounds 11 and 12. Data are presented as mean±S.E.M. (n=3). nNH, number of hydrogen bond donors; nON, number of hydrogen bond acceptors; MW, molecular weight; TPSA, topological polar surface area; nRot. bond, number of rotatable bonds; MIC, minimum inhibitory concentration. ^{a}Calculated using molinspiration online service; ^{b}Calculated using ChemDraw Ultra 13.0, CambridgeSoft.

FIGS. 3A-3B show that MmpL3 is a validated target in Mycobacterium tuberculosis. (a) Illustration of the topology of the MmpL3 mycolic acid transporter protein in the M. tuberculosis inner membrane. Colored circles represent the locations of amino acid changes associated with resistance to compounds known to target this protein: the diamide SQ109³, the pyrrole derivative BM212^{4,5}, and the urea derivative AU1235⁶. (b) Structures of BM212, AU1235 and 5Q109.

FIG. 4 shows that indoleamide compound 12 is active against Mycobacterium tuberculosis in a dose-dependent manner during in vivo infection of BALB/c mice. Lung CFU counts were assessed 4 weeks after starting daily oral administration of compound 12. Each dot represents CFUs from the lungs of an individual mouse, and the bars indicate mean±S.D. CFU counts in each group (n=5 for treated groups and n=4 for untreated control because of one accidental death prematurely). Statistical significance was assessed using the one-way ANOVA with Tukey's multiple comparison test. CFU, colony forming unit.

FIGS. 5A-5B show the pharmacokinetic analysis of compound 12 in female BALB/c mice. (a) Concentration in plasma and (b) concentration in lung following a single 100 mg/kg dose administered by oral gavage. Data are presented as mean±S.E.M. (n=3).

FIG. 6 shows the serum inhibition titration result for compound 1y (N-(2,3,5 -methyl, 4-dimethyl)-4,6,-difluoro-1H-indole-2-carboxamide). Compound 1y was administered at 100 mg/kg to Balb/c mice by oral gavage using the vehicle 0.5% CMC. After 30, 60, and 120 min, blood was collected. The mouse sera were serially diluted, and 10000 CFUs of Mtb were added per well. The inhibition at end point was monitored by the alarmar Blue assay and plotted as relative fluorescence units. Isoniazid (INH) was included as a positive control.

### DETAILED DESCRIPTION OF THE INVENTION

### Definitions

The following terms and expressions used herein have the indicated meanings.

“Alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, hexyloxy, and the like.

“Alkyl” means a straight or branched chain hydrocarbon containing from 1 to 12 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

“Amino” means a group of formula NRₚR_{ct} where Rₚ and R_{ct} are independently selected from H and C₁-C₄ alkyl. Representative amino include amino (NH₂), methylamino, dimethylamino, diisopropylamino, dibutylamino, and the like.

“Aryl,” means a phenyl (i.e., monocyclic aryl containing only carbon atoms in the aromatic ring system. The aryl may be unsubstituted or substituted with one or more alkyl, alkoxy, halo, halolakyl or amino groups.

“Cycloalkyl” means a monocyclic or a bicyclic cycloalkyl ring system. Monocyclic ring systems are cyclic hydrocarbon groups containing from 3 to 10 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In certain embodiments of the disclosure, the cycloalkyl is cyclopentyl, cyclohexyl, cycloheptyl, or cyclooctyl. The cyclolalkyl may be unsubstituted or substituted with one or more alkyl, alkoxy, halo, halolakyl or amino groups.

“Halo” or “halogen” means —Cl, —Br, —I or —F.

“Haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Heteroaryl” means a monocyclic ring system containing a 5- or 6-membered heteroaromatic ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The heteroaryl may be unsubstituted or substituted with one or more alkyl, alkoxy, halo, halolakyl or amino groups.

“Heterocyclyl” as used herein, means a monocyclic 5- or 6-membered ring containing at least one heteroatom independently selected from the group consisting of O, N, and S where the ring is saturated or unsaturated, but not aromatic. The 5-membered ring can contain zero or one double bond and one, two or three heteroatoms selected from the group consisting of O, N and S. The 6-membered ring can contain zero, one or two double bonds and one, two or three heteroatoms selected from the group consisting of O, N and S. The heterocyclyl is connected to the parent molecular moiety through any carbon atom or any nitrogen atom contained within the heterocyclyl. Representative heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl may be unsubstituted or substituted with one or more alkyl, alkoxy, halo, halolakyl or amino groups.

“Saturated” means the referenced chemical structure does not contain any multiple carbon-carbon bonds. For example, a saturated cycloalkyl group as defined herein includes cyclohexyl, cyclopropyl, and the like.

“Unsaturated” means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

“Pharmaceutically acceptable salt” refers to both acid and base addition salts.

“Modulating” or “modulate” refers to the treating, prevention, suppression, enhancement or induction of a function, condition or disorder. For example, it is believed that the compounds of the present disclosure can modulate atherosclerosis by stimulating the removal of cholesterol from atherosclerotic lesions in a human.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, or slowing progression of one or more symptoms of the disease or disorder

“Subject” refers to a warm blooded animal such as a mammal, preferably a human, or a human child, which is afflicted with, or has the potential to be afflicted with one or more diseases and disorders described herein.

This invention is a series of indoleamides and analogs having potent activity against both drug-susceptible and drug-resistant strains of M. tuberculosis.

In its principle aspect, this invention is a compound of formula I:

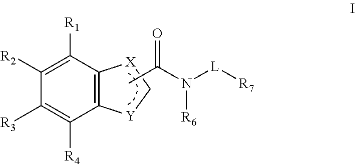

Wherein R₁, R₂, R₃ and R₄ are independently selected from H, alkyl, haloalkyl, alkoxy, halo and amino; X is CH or S; Y is O or NR₅; R₆ is H or alkyl; R₇ is C₃-C₁₀ cycloalkyl, C₆ aryl, or alkyl; and R₅ is H or alkyl, or a pharmaceutically acceptable salt, solvate, or stereoisomer thereof.

In an embodiment, L is absent or CH₂.

In another embodiment, Y is NR₅ wherein R₅ is H.

In another embodiment, R₂ and R₄ are H.

In another embodiment, R₂ and R₄ are H and R₁ and R₃ are methyl or halogen.

In another embodiment, R₂ and R₄ are H and R₁ and R₃ are H or halogen.

In another embodiment, R₇ is a C₆ cycloalkyl and R₂ and R₄ are H and R₁ and R₃ are H or halogen.

In another embodiment, R₇ is C₈-C₁₀ cycloalkyl, C₅-C₈ heterocyclyl or C₅-C₆ heteroaryl.

In another embodiment, R₇ is C₈-C₁₀ cycloalkyl.

In another embodiment, this invention is a compound of formula:

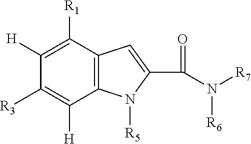

wherein R₁ and R₃ are Cl or F and R₇ is C₈-C₁₀ cycloalkyl.

In another embodiment, this invention is a compound of formula:

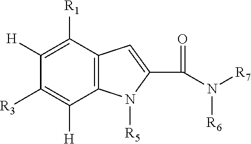

wherein R₁ and R₃ are independently Br or F and R₇ is C₅-C₈ cycloalkyl.

In another embodiment, this invention is a compound of formula

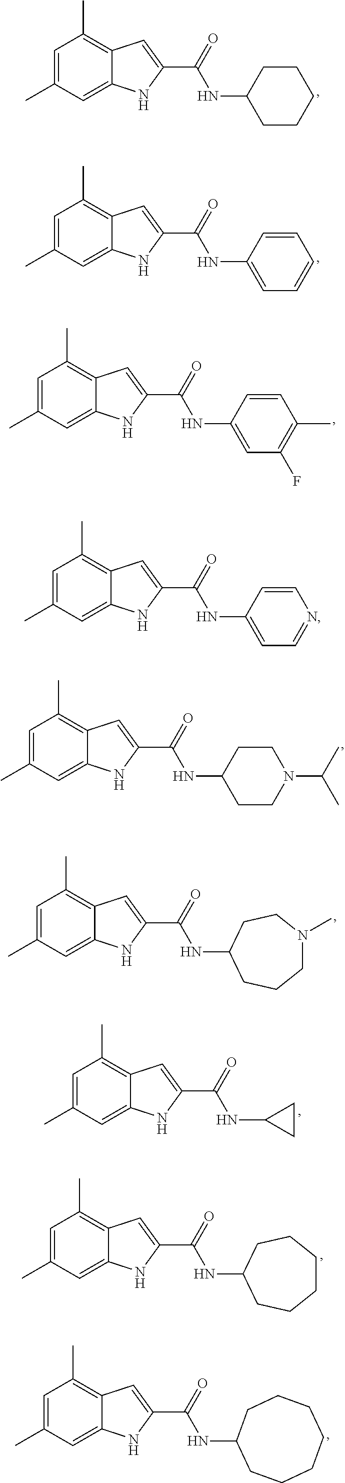

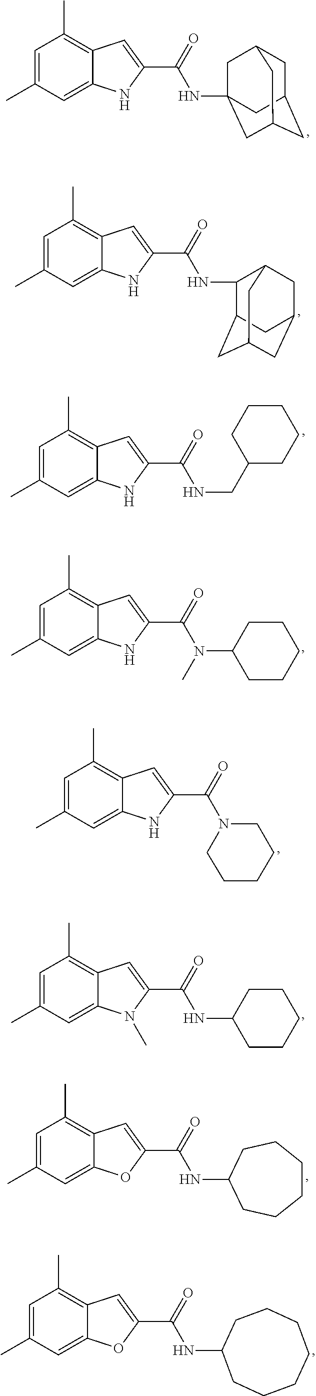

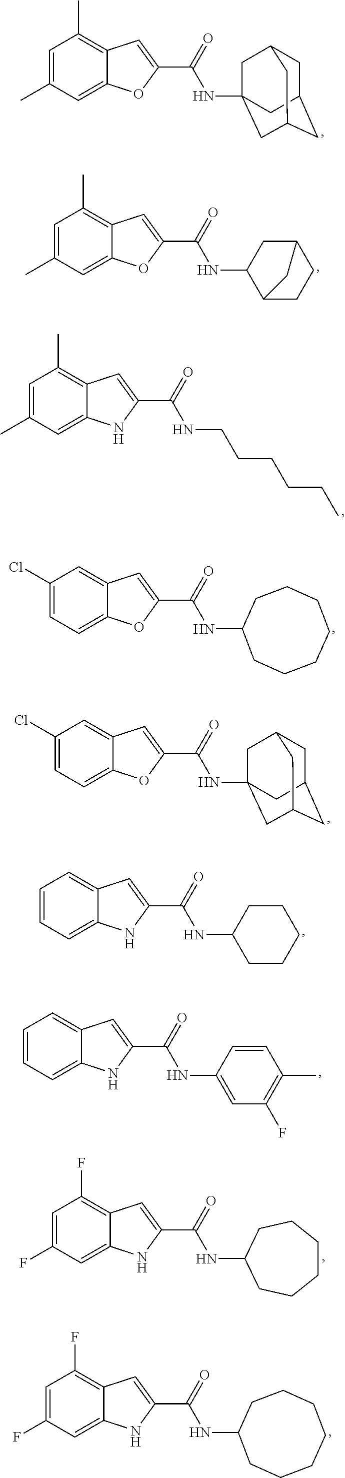

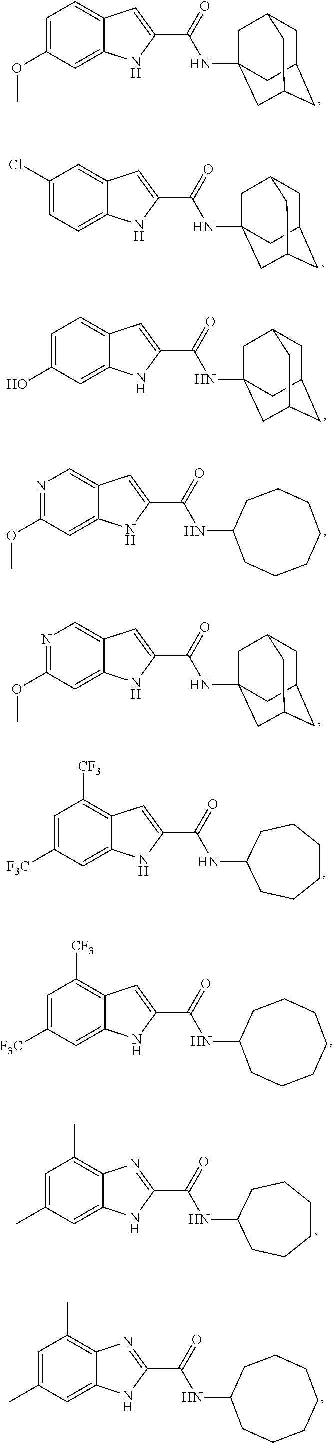

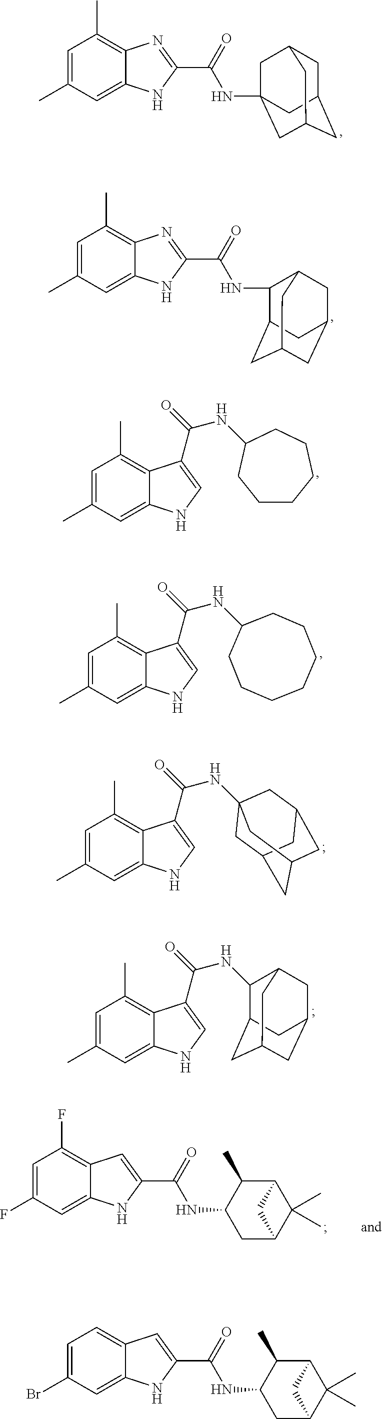

or a pharmaceutically acceptable salt, solvate, or stereoisomer thereof.

In another embodiment, this invention is a compound of formula

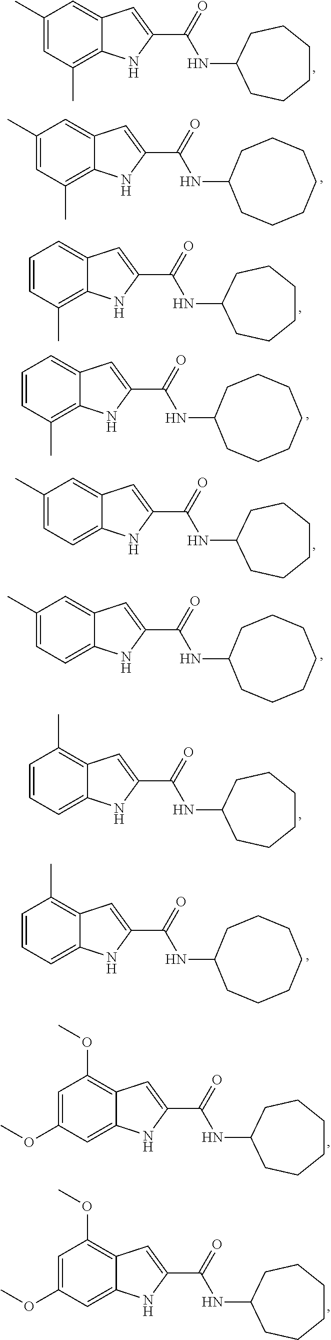

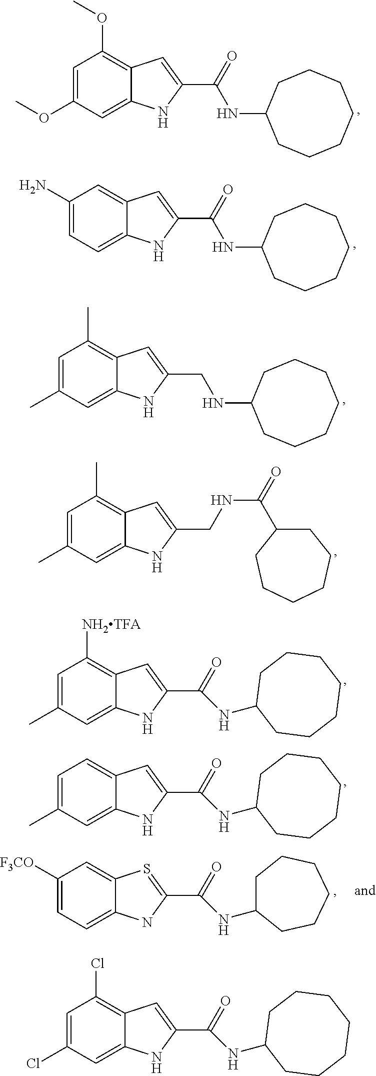

or a pharmaceutically acceptable salt thereof.

In another embodiment, this invention is a compound according of formula

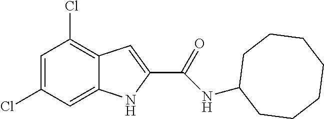 [SMILES: O=C(NC1CCCCCCC1)c1cc2c(Cl)cc(Cl)cc2[nH]1]

or a pharmaceutically acceptable salt thereof.

In other aspects, the disclosure provides a pharmaceutical composition comprising a therapeutically effective amount of a compound of formula I as described herein, and one or more pharmaceutically acceptable diluents, preservatives, solubilizers, emulsifiers, adjuvants, excipients, or carriers. The pharmaceutical composition can be used, for example, treating tuberculosis in a subject in need thereof. In certain embodiments, the tuberculosis is MDR or XDR tuberculosis.

In certain embodiments, this invention is a pharmaceutical composition comprising a compound of formula I together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, and the like.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

In accordance with some embodiments, the present invention provides a composition comprising one or more compounds of formula I and at least one or more additional biologically active agents, and a pharmaceutically acceptable carrier.

In some embodiments, the biologically active agents are anti-infective agents. Examples of such anti-infective agents include, anti-infective agents, such as antihelmintics, antianaerobics, antibiotics, aminoglycoside antibiotics, antifungal antibiotics, cephalosporin antibiotics, macrolide antibiotics, miscellaneous antibiotics, penicillin antibiotics, quinolone antibiotics, sulfonamide antibiotics, tetracycline antibiotics, antimycobacterials, antituberculosis and antimycobacterials, such as isoniazid and rifampin.

“Pharmaceutically acceptable vehicle” means a diluent, adjuvant, excipient or carrier with which a compound of the disclosure is administered. The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

“Pharmaceutically acceptable carriers” for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the invention will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the invention can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

Yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The foregoing may be better understood by reference to the following Experimental section, which is presented solely for purposes of illustration and is not intended to limit the scope of the invention.

### EXPERIMENTAL

The hit compound 3 obtained from high throughput screening (HTS) was resynthesized to confirm the activity along with 40 novel derivatives (4-44) employing an efficient amide coupling protocol (Scheme 1 and 2). Briefly, following a Fischer indole synthesis protocol, 3,5-dimethylphenylhydrazine hydrochloride (45) was reacted with ethyl pyruvate under acidic conditions to afford the disubstituted indole-2-carboxylate 46, and subsequent basic hydrolysis afforded the corresponding acid 47. N-methylation of 46 followed by basic hydrolysis gave the carboxylic acid 48. The carboxylic acids were subsequently reacted with their corresponding amine in the presence of 1-ethyl-3-(3-dimethylaminopropyl)carbodiimide hydrochloride (EDC.HCl) and hydroxybenzotriazole (HOBt) as coupling agents and triethylamine as a base to obtain compounds 3-18 (Scheme 1). 5-Chlorobenzofuran-2-carboxylic acid (53)⁷ and 4,6-dimethylbenzofuran-2-carboxylic acid (54)⁸ were obtained from the starting materials 49 and 50 via intermediates 52 and 51, respectively, following a modified literature protocol (Scheme 1).^{9,10} Compound 54 was reacted with the appropriate amines under standard amide coupling conditions to obtain compounds 19-23. Following similar conditions, compounds 24 and 25 were obtained by reacting carboxylic acid 53 with the appropriate amines (Scheme 1).

The unsubstituted and monosubstituted carboxylic acids (55-59) were reacted with their corresponding amines to afford compounds 26-31,33 and 34 while compound 32 was obtained from its methoxy precursor 30 using boron tribromide (Scheme 2). 3,5-Bis(trifluoromethyl)phenylhydrazine hydrochloride (60) was reacted with ethyl pyruvate under microwave irradiation to obtain its hydrazone intermediate 61, which was further subjected to acidic conditions to obtain the cyclized indole (62). Basic hydrolysis then afforded the desired carboxylic acid 63. Compound 63 was reacted with cycloheptylamine or cyclooctylamine to provide the amides 35 and 36. 3,5-Dimethylbenzene-1,2-diamine (64) was reacted with methyl-2,2,2-trichloroacetimidate to afford its trichloromethyl intermediate 65, followed by basic hydrolysis to give the corresponding acid 66. Decarboxylation of indole 47 with copper powder in quinoline gave the desired intermediate 67, which was subsequently reacted with trichloroacetyl chloride to give the trichloromethyl intermediate 68. Subsequent basic hydrolysis afforded 4,6-dimethyl-1H-indole-3-carboxylic acid (69). Compounds 66 and 69 were reacted with their corresponding amines under standard amide coupling conditions to obtain compounds 37-44 (Scheme 2).

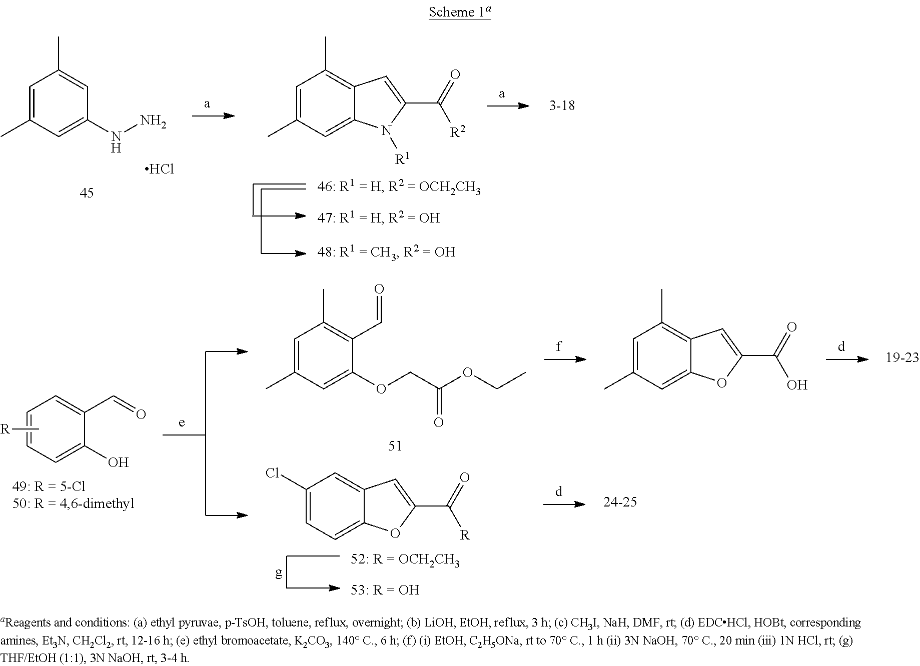

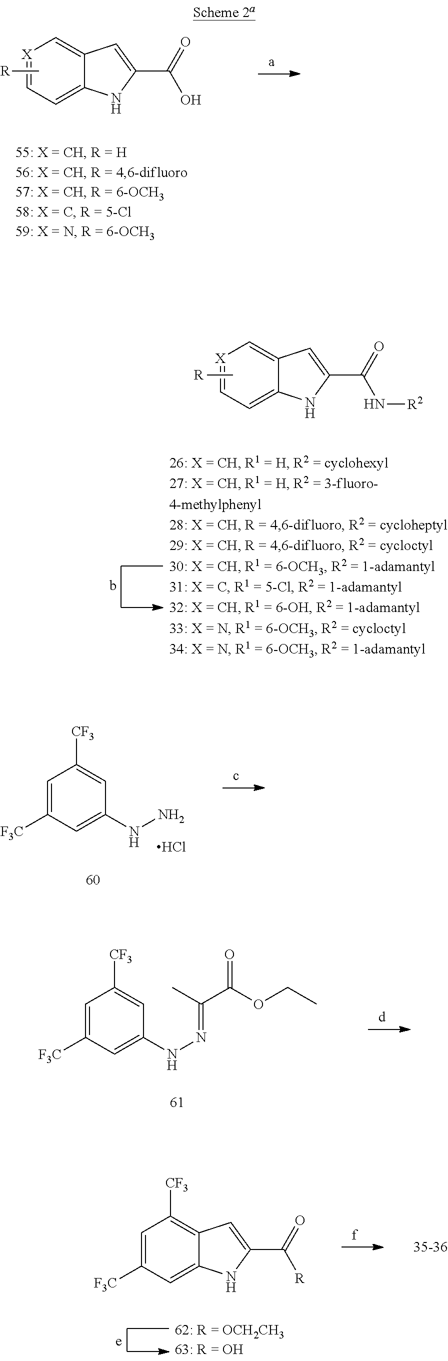

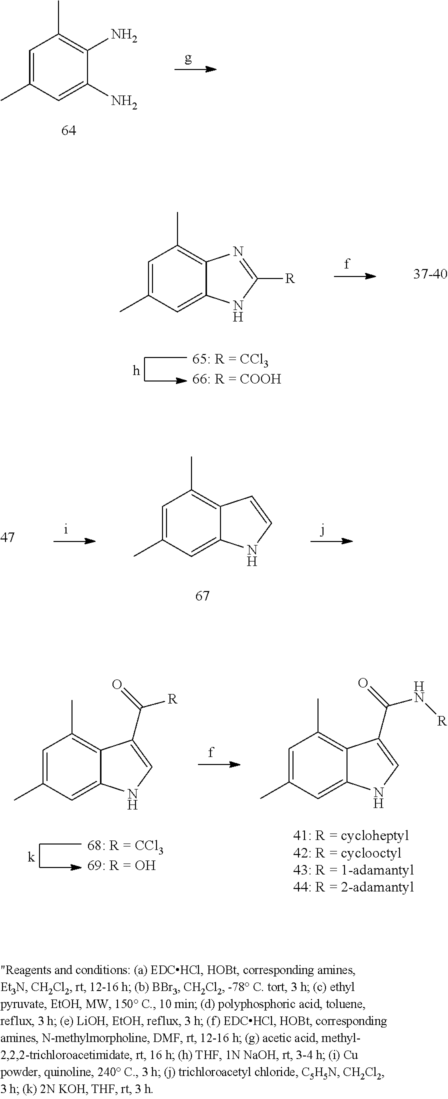

### General Information.

The following carboxylic acids, 1H-indole-2-carboxylic acid, 5-chloro-1H-indole-2-carboxylic acid, 6-methoxy-1H-indole-2-carboxylic acid, were purchased from Sigma-Aldrich while 6-methoxy-1H-pyrrolo[3,2-c]pyridine-2-carboxylic acid and 4,6-difluoro-1H-indole-2-carboxylic acid were purchased from Chem-Impex and Combi-blocks. Anhydrous dichloromethane (CH₂Cl₂) was obtained by distillation over calcium hydride. ¹H NMR and ¹³C NMR spectra were recorded on a Bruker spectrometer at 400 MHz and 100 MHz, respectively, with TMS as an internal standard. Standard abbreviation indicating multiplicity was used as follows: s=singlet, d=doublet, dd=doublet of doublets, t=triplet, q=quadruplet, m=multiplet and br=broad. HRMS experiments were performed on Q-TOF-2TM (Micromass) and IT-TOF (Shimadzu) instruments. TLC was performed with Merck 60 F254 silica gel plates. Flash chromatography was performed using CombiFlash0 Rf system with RediSep® columns or alternatively using Merck silica gel (40-60 mesh). Final compounds were purified by preparative HPLC unless otherwise stated. The preparative HPLC employed an ACE 5-AQ (21.2 mm×150 mm) column, with detection at 254 and 280 nm on a Shimadzu SCL-10A VP detector, flow rate=17.0 mL/min. Method 1: 50-100% CH₃OH/H₂O in 30 min; 100% CH₃OH in 5 min; 100-50% CH₃OH/H₂O in 4 min. Method 2: 25-100% CH₃OH/H₂O in 30 min; 100% CH₃OH in 5 min; 100-25% CH₃OH/H₂O in 4 min. Method 3: 15-100% CH₃OH/H₂O in 30 min; 100% CH₃OH in 5 min; 100-15% CH₃OH/H₂O in 4 min. Both solvents contains 0.05 vol % of trifluoroacetic acid (TFA). Purities of final compounds were established by analytical HPLC, which was carried out using the Agilent 1100 HPLC system with a Synergi 4 μm Hydro-RP 80A column, on a variable wavelength detector G1314A. Method 1: flow rate=1.4 mL/min; gradient elution over 20 minutes, from 30% Me0H-H₂O to 100% Me0H with 0.05% TFA. Method 2: flow rate=1.4 mL/min; gradient elution over 20 minutes, from 50% Me0H-H₂O to 70% Me0H with 0.05% TFA. The purity of all tested compounds was >95% as determined by the method described above.

### General Procedure for the Synthesis of 3-44

To a solution of the appropriate carboxylic acid (1 equiv) in anhydrous dichloromethane (CH₂Cl₂) or dimethylformamide (DMF) (4 mL/mmol) at room temperature were added anhydrous hydroxybenzotriazole (HOBt, 1 equiv) and 1-ethyl-3-(3-dimethylaminopropyl)carbodiimide hydrochloride (EDC.HCl, 1 equiv) under an argon atmosphere. After stirring for 10 min, the appropriate substituted amine (1 equiv) and triethylamine or N-methyl morpholine (1.5 equiv) were added, and the reaction mixture was stirred at room temperature until disappearance of the starting material (usually 12 to 16 h). After this time water (2 mL) was added, and the mixture was extracted with EtOAc (3×10 mL), the organic layers were separated, washed with brine, dried over anhydrous Na₂SO₄, filtered, and concentrated under reduced pressure. The residue was purified by flash chromatography (EtOAchexane 1:4 unless specified differently) to obtain the indoleamides in yields ranging from 34 to 95%.

N-Cyclohexyl-4,6-dimethyl-1H-indole-2-carboxamide (3).

Yield 92% (white powder). ¹H NMR (400 MHz, CDCl₃) δ9.32 (br s, 1H), 7.07 (s, 1H), 6.79 (s, 2H), 6.03 (d, J=7.6 Hz, 1H), 4.07-3.98 (m, 1H), 2.44 (s, 3H), 2.37 (s, 3H), 2.10-2.06 (m, 2H), 1.83-1.78 (m, 2H), 1.68-1.45 (m, 2H), 1.31-1.26 (m, 4H); ¹³C NMR (100 MHz, CDCl₃) δ160.9, 136.5, 134.6, 130.9, 129.9, 125.7, 122.7, 109.2, 99.9, 48.5, 33.3, 25.6, 24.9, 21.8, 18.6. HRMS (ESI) calcd for C₁₇H₂₂N₂O ([M+H]⁺) 271.1805; found: 271.1809.

N-Phenyl-4,6-dimethyl-1H-indole-2-carboxamide (4).

Yield 67% (white powder). ¹H NMR (400 MHz, CDCl₃) δ9.47 (br s, 1H), 7.89 (br s, 1H), 7.70 (d, J=8.0 Hz, 2H), 7.42 (t, J=7.6 Hz, 2H), 7.19 (t, J=7.6 Hz, 1H), 7.09 (s, 1H), 7.00 (s, 1H), 6.83 (s, 1H), 2.56 (s, 3H), 2.45 (s, 3H); ¹³C NMR (100 MHz, CDCl₃) δ159.9, 139.2, 137.2, 133.3, 130.3, 128.7, 125.3, 123.4, 122.0, 120.0, 109.6, 102.7, 99.6, 21.6, 18.5. HRMS (ESI) calcd for C₁₇H₁₆N₂O ([M+H]⁺) 265.1335; found: 265.1348

N-(3-Fluoro-4-methylphenyl)-4,6-dimethyl-1H-indole-2-carboxamide (5).

Yield 89% (white powder). ¹H NMR (400 MHz, d₆-DMSO) δ11.61 (s, 1H), 10.26 (s, 1H), 7.80 (d, J=12.4 Hz, 1H), 7.52 (d, J=8.4 Hz, 1H), 7.45 (s, 1H), 7.24 (t, J=8.8 Hz, 1H), 7.11 (s, 1H), 6.70 (s, 1H), 2.50 (s, 3H), 2.21 (s, 3H), 1.98 (s, 3H); ¹³C NMR (100 MHz, d₆-DMSO) δ161.5 (J=239 Hz), 159.9, 138.6 (J=11 Hz), 137.3, 133.4, 131.3 (J=6.3 Hz), 130.3, 130.0, 125.3, 122.0, 118.6 (J=17.2 Hz), 115.5, 109.6, 106.7 (J=27 Hz), 102.8, 21.5, 18.4, 13.7 (J=2.9 Hz). HRMS (ESI) calcd for C₁₈H₁₇FN₂O ([M+H]⁺) 297.1252; found: 297.1266.

N-(4-Pyridinyl)-4,6-dimethyl-1H-indole-2-carboxamide (6).

Yield 77% (white powder). ¹H NMR (400 MHz, CD₃OD) δ8.43 (d, J=5.2 Hz, 2H), 7.88 (d, J=6.3 Hz, 2H), 7.43 (s, 1H), 7.27 (s, 1H), 6.87 (s, 1H), 2.54 (s, 3H), 2.42 (s, 3H); ¹³C NMR (100 MHz, d₆-DMSO) δ160.6, 150.4, 145.9, 137.6, 133.9, 130.6, 129.5, 125.2, 122.3, 113.7, 109.7, 103.8, 99.6, 21.6, 18.5. HRMS (ESI) calcd for Cᵢ₆H₁₅N₃O ([M+H]⁺) 266.1288; found: 266.1295.

N-(1-Methyl-4-piperidinyl)-4,6-dimethyl-1H-indole-2-carboxamide (7).

Purified by column chromatography (EtOAc-hexane 1:1). Yield 65% (white powder). ¹H NMR (400 MHz, d₆-DMSO) δ11.24 (s, 1H), 8.18 (d, J=8 Hz, 1H), 7.13 (s, 1H), 7.02 (s, 1H), 6.58 (s, 1H), 3.76-3.70 (m, 1H), 2.78-2.75 (m, 2H), 2.43 (s, 3H), 2.33 (s, 3H), 2.16 (s, 3H), 2.00-1.91 (m, 2H), 1.77 (m, 2H), 1.65-1.47 (m, 2H); ¹³C NMR (100 MHz, d₆-DMSO) δ160.6, 136.7, 132.5, 130.7, 129.9, 125.3, 121.7, 109.5, 101.4, 54.6, 46.1, 31.7, 21.6, 18.5. HRMS (ESI) calcd for C₁₇H₂₃N₃O ([M+H]⁺) 286.1914; found: 286.1908.

N-(1-Isopropyl-4-piperidinyl)-4,6-dimethyl-1H-indole-2-carboxamide (8).

Yield 70% (white powder). ¹H NMR (400 MHz, d₆-DMSO) δ11.43 (s, 1H), 8.15 (d, J=8.0 Hz, 1H), 7.13 (s, 1H), 7.02 (s, 1H), 6.66 (s, 1H), 3.74-3.72 (m, 1H), 2.81-2.78 (m, 2H), 2.70 (m, 1H), 2.43 (s, 3H), 2.33 (s, 3H), 2.17 (t, J=12.0 Hz, 2H), 1.97-1.81 (m, 2H), 1.56-1.48 (m, 2H), 0.97 (d, J=6.4 Hz, 6H); ¹³C NMR (100 MHz, d₆-DMSO) δ160.5, 136.7, 132.5, 130.7, 129.9, 125.3, 121.7, 109.5, 101.3, 53.7, 47.4, 47.0, 32.2, 21.6, 18.5, 18.2. HRMS (ESI) calcd for C₁₉H₂₇N₃O ([M+H]⁺) 314.2227; found: 314.2216.

N-(1-Methyl-4-azepanyl)-4,6-dimethyl-1H-indole-2-carboxamide (9).

Yield 83% (white powder). ¹H NMR (400 MHz, d₆-DMSO) δ11.37 (s, 1H), 9.50 (br s, 1H), 8.11 (d, J=8.0 Hz, 1H), 7.11 (s, 1H), 7.00 (s, 1H), 6.57 (s, 1H), 4.17 (br s, 1H), 3.00-3.70 (m, 4H), 2.82 (s, 3H), 2.47 (s, 3H), 2.33 (s, 3H), 2.07-1.68 (m, 6H); ¹³C NMR (100 MHz, d₆-DMSO) δ160.4, 136.7, 132.7, 130.4, 130.0, 125.2, 121.8, 109.5, 101.5, 52.5, 48.1, 47.1, 43.8, 32.2, 21.6, 18.5. HRMS (ESI) calcd for C₁₈H₂₅N₃O ([M+H]⁺) 300.2070; found: 300.2068.

N-Cyclopropyl-4,6-dimethyl-1H-indole-2-carboxamide (10).

Yield 95% (white powder). ¹H NMR (400 MHz, CDCl₃) δ9.47 (br s, 1H), 7.07 (s, 1H), 6.79 (s, 2H), 6.43 (s, 1H), 3.11-2.92 (m, 1H), 2.50 (s, 3H), 2.43 (s, 3H), 0.94-0.88 (m, 2H), 0.76-0.67 (m, 2H); ¹³C NMR (100 MHz, CDCl₃) δ162.9, 137.1, 133.0, 130.9, 130.3, 125.7, 122.1, 109.9, 101.7, 23.1, 21.9, 18.8, 6.2. HRMS (ESI) calcd for C₁₄H₁₆N₂O ([M+H]⁺) 229.1335; found: 229.1342.

N-Cycloheptyl-4,6-dimethyl-1H-indole-2-carboxamide (11).

Yield 72% (white powder). ¹H NMR (400 MHz, CD₃OD) δ7.12 (s, 1H), 7.05 (s, 1H), 6.70 (s, 1H), 4.09-4.04 (m, 1H), 2.48 (s, 3H), 2.38 (s, 3H), 2.00-1.98 (m, 2H), 1.76-1.54 (m, 10H); ¹³C NMR (100 MHz, CDCl₃) δ159.8, 135.2, 131.8, 128.5, 127.9, 123.7, 119.8, 106.9, 99.8, 48.9, 32.7, 25.8, 22.2, 18.6, 15.4. HRMS (ESI) calcd for C₁₈H₂₄N₂O ([M+H]⁺) δ85.1889; found: 285.1892.

N-Cyclooctyl-4,6-dimethyl-1H-indole-2-carboxamide (12).

Yield 83% (white powder). ¹H NMR (400 MHz, d₆-DMSO) δ11.29 (s, 1H), 8.14 (d, J=8.0 Hz, 1H), 7.16 (d, J=1.6 Hz, 1H), 7.02 (s, 1H), 6.66 (s, 1H), 4.06-4.01 (m, 1H), 2.44 (s, 3H), 2.34 (s, 3H), 1.81-1.65 (m, 6H), 1.61-1.51 (m, 8H); ¹³C NMR (100 MHz, d₆-DMSO) δ158.9, 134.4, 131.1, 127.7, 127.1, 122.6, 119.0, 106.1, 98.9, 46.8, 29.4, 24.0, 22.7, 21.0, 17.8, 14.6. HRMS (ESI) calcd for C₁₉H₂₆N₂O ([M+H]⁺) 299.2117; found: 299.2115.

N-(1-Adamantyl)-4,6-dimethyl-1H-indole-2-carboxamide (13).

Yield 65% (white powder). ¹H NMR (400 MHz, d₆-DMSO) δ11.21 (s, 1H), 7.14 (s, 1H), 7.00 (s, 1H), 6.65 (s, 1H), 2.42 (s, 3H), 2.33 (s, 3H), 2.09 (s, 6H), 2.07 (br s, 3H), 1.67 (s, 6H); ¹³C NMR (100 MHz, d₆-DMSO) δ161.1, 136.9, 132.8, 131.8, 130.3, 125.7, 122.0, 109.8, 101.9, 51.9, 41.6, 36.5, 29.3, 21.9, 18.9. HRMS (ESI) calcd for C₂₁H₂₆N₂O ([M+H]⁺) 323.2117; found: 323.2105.

N-(2-Adamantyl)-4,6-dimethyl-1H-indole-2-carboxamide (14).

Purified by re-crystallization from EtOH-Et₂O. Yield 82% (white powder). ¹H NMR (400 MHz, d₆-DMSO) δ11.30 (s, 1H), 7.72 (d, J=6.8 Hz, 1H), 7.32 (d, J=1.6 Hz, 1H), 7.02 (s, 1H), 6.66 (s, 1H), 4.09 (d, J=5.2 Hz, 1H), 2.45 (s, 3H), 2.34 (s, 3H), 2.14 (d, J=12.4 Hz, 2H), 1.98 (s, 2H), 1.86-1.83 (m, 6H), 1.73 (s, 2H), 1.54 (d, J=12.0 Hz, 2H); ¹³C NMR (100 MHz, d₆-DMSO) δ160.8, 136.7, 132.6, 130.5, 130.0, 125.3, 121.6, 109.4, 102.2, 53.6, 37.2, 36.9, 31.4, 31.1, 26.8, 21.5, 18.4. HRMS (ESI) calcd for C₂₁H₂₆N₂O ([M+H]⁺) 323.2117; found: 323.2113.

N-(Cyclohexylmethyl)-4,6-dimethyl-1H-indole-2-carboxamide (15).

Yield 80% (white powder). ¹H NMR (400 MHz, CDCl₃) δ9.41 (s, 1H), 7.07 (s, 1H), 6.82 (d, J=8.9 Hz, 2H), 6.26 (m, 1H), 3.36 (t, J=6.5 Hz, 2H), 2.53 (s, 3H), 2.44 (s, 3H), 1.85-1.62 (m, 6H), 1.30-1.21 (m, 3H), 1.08-1.02 (m, 2H); ¹³C NMR (100 MHz, CDCl₃) δ161.5, 136.2, 134.2, 130.5, 129.3, 125.3, 122.4, 108.8, 99.7, 45.4, 37.8, 30.5, 26.0, 25.4, 21.4, 18.2. HRMS (ESI) calcd for C₁₈H₂₄N₂O ([M+H]⁺) 285.1889; found: 285.1967.

N-Cyclohexyl-N,4,6-trimethyl-/H-indole-2-carboxamide (16).

Yield 88% (white powder). ¹H NMR (400 MHz, d₆-DMSO) δ11.33 (s, 1H), 7.05 (s, 1H), 6.74-6.67 (m, 2H), 4.33 (m, 1H), 3.07 (br s, 3H), 2.45 (s, 3H), 2.35 (s, 3H), 1.81-1.56 (m, 7H), 1.34-1.31 (m, 2H), 1.18-1.10 (m, 1H); ¹³C NMR (100 MHz, d₆-DMSO) δ162.6, 136.0, 132.5, 129.9, 129.4, 125.1, 121.7, 109.2, 29.6, 25.3, 24.9, 21.5, 18.3. HRMS (ESI) calcd for C₁₈H₂₄N₂O ([M+H]⁺) 285.1961; found: 285.1969.

(4,6-Dimethyl-11-1-indo1-2-yl)(piperidin-1-yl)methanone (17).

Recrystallization from EtO-Et₂O. Yield 93% (white powder). ¹H NMR (400 MHz, d₆-DMSO) δ11.32 (s, 1H), 7.01 (s, 1H), 6.68 (d, J=1.2 Hz, 1H), 6.66 (s, 1H), 3.71 (br s, 4H), 2.43 (s, 3H), 2.34 (s, 3H), 1.66-1.64 (m, 2H), 1.57-1.56 (m, 4H); ¹³C NMR (100 MHz, d₆-DMSO) δ162.5, 136.5, 132.8, 130.3, 129.5, 125.4, 122.1, 109.6, 102.5, 26.3, 24.6, 21.9, 18.8. HRMS (ESI) calcd for Cᵢ₆H₂₀N₂O ([M+H]⁺) 257.1648; found: 257.1652.

N-Cyclohexyl-1,4,6-trimethyl-11-1-indole-2-carboxamide (18).

Yield 74% (white powder). ¹H NMR (400 MHz, CDCl₃) δ7.01 (s, 1H), 6.79 (d, J=8.0 Hz, 2H), 6.06 (d, J=7.6 Hz, 1H), 4.02 (s, 3H), 3.99-3.93 (m, 1H), 2.52 (s, 3H), 2.48 (s, 3H), 2.08-2.05 (m, 2H), 1.82-1.66 (m, 3H), 1.51-1.40 (m, 2H), 1.33-1.19 (m, 3H); ¹³C NMR (100 MHz, CDCl₃) δ161.5, 138.9, 133.8, 130.9, 130.4, 123.6, 122.2, 107.1, 101.4, 47.9, 32.9, 31.2, 25.2, 24.6, 21.7, 18.1. HRMS (ESI) calcd for C₁₈H₂₄N₂O ([M+H]⁺) 285.1889; found: 285.1974.

N-Cycloheptyl-4,6-dimethylbenzofuran-2-carboxamide (19).

Purified by flash chromatography (CH₂Cl₂, 100%). Yield 83% (off-white solid). ¹H NMR (400 MHz, d₆-DMSO) δ8.39 (d, J=8.0 Hz, 1H), 7.55 (s, 1H), 7.23 (s, 1H), 6.94 (s, 1H), 3.96 (m, 1H), 2.46 (s, 3H), 2.40 (s, 3H), 1.86-1.82 (m, 2H), 1.65-1.41 (m, 10H); ¹³C NMR (100 MHz, d₆-DMSO) δ157.1, 154.5, 148.4, 136.6, 131.7, 125.2, 124.7, 109.0, 107.9, 50.1, 34.2, 27.7, 23.9, 21.4, 18.1. HRMS (ESI) calcd for C₁₈H₂₃NO₂([M+H]⁺) 286.1802; found: 286.1813.

N-Cyclooctyl-4,6-dimethylbenzofuran-2-carboxamide (20).

Purified by flash chromatography (CH₂Cl₂/CH₃OH, 9:1). Yield 69% (off-white solid). ¹H NMR (400 MHz, d₆-DMSO) δ8.37 (d, J=8.1 Hz, 1H), 7.55 (s, 1H), 7.24 (s, 1H), 6.95 (s, 1H), 4.02 (m, 1H), 2.46 (s, 3H), 2.40 (s, 3H), 1.72-1.69 (m, 6H), 1.62-1.50 (m, 8H); ¹³C NMR (100 MHz, d₆-DMSO) δ157.1, 154.5, 148.4, 136.6, 131.7, 125.2, 124.7, 109.0, 107.9, 48.9, 31.6, 26.8, 25.1, 23.5, 21.4, 18.1. HRMS (ESI) calcd for C₁₉H₂₅NO₂([M+Na]⁺) 322.1778; found: 322.1786.

N-(1-Adamantyl)-4,6-dimethylbenzofuran-2-carboxamide (21).

Purified by flash chromatography (CH₂Cl₂/CH₃OH, 9:1). Yield 72% (off-white solid). ¹H NMR (400 MHz, d₆-DMSO) δ7.57 (s, 1H), 7.54 (s, 1H), 7.23 (s, 1H), 6.94 (s, 1H), 2.45 (s, 3H), 2.39 (s, 3H), 2.08 (br s, 9H), 1.66 (m, 6H); ¹³C NMR (100 MHz, d₆-DMSO) δ157.6, 154.4, 148.5, 136.5, 131.7, 125.2, 124.7, 109.0, 107.8, 51.7, 40.8, 36.0, 28.8, 21.4, 18.1. HRMS (ESI) calcd for C₂₁H₂₅NO₂ ([M+H]⁺) 324.1958; found: 324.1966.

N-(bicyclo[2.2.1]-2-heptanyl)-4,6-dimethylbenzofuran-2-carboxamide (22).

Purified by flash chromatography (CH₂Cl₂, 100%). Yield 82% (off-white solid). ¹H NMR (400 MHz, d₆-DMSO) δ8.24 (d, J=6.7 Hz, 1H), 7.59 (s, 1H), 7.24 (s, 1H), 6.94 (s, 1H), 3.72 (m, 1H), 2.46 (s, 3H), 2.40 (s, 3H), 2.24 (s, 1H), 2.18 (d, J=2.4 Hz, 1H), 1.66-1.40 (m, 5H), 1.22-1.09 (m, 3H); ¹³C NMR (100 MHz, d₆-DMSO) δ157.7, 154.5, 148.2, 136.6, 131.7, 125.2, 124.7, 109.0, 108.0, 52.6, 42.0, 37.9, 35.2, 34.9, 28.0, 26.3, 21.4, 18.1. HRMS (ESI) calcd for Cᵢ₈H₂ᵢNO₂([M+H]⁺) 284.1645; found: 284.1644.

N-Hexyl-4,6-dimethylbenzofuran-2-carboxamide (23).

Purified by flash chromatography (CH₂Cl₂/CH₃OH, 9:1). Yield 74% (pale yellow solid). ¹H NMR (400 MHz, d₆-DMSO) δ8.57 (t, J=5.6 Hz, 1H), 7.51 (s, 1H), 7.23 (s, 1H), 6.95 (s, 1H), 3.24 (m, 2H), 2.46 (s, 3H), 2.40 (s, 3H), 1.53-1.48 (m, 2H), 1.27 (br s, 6H), 0.86 (t, J=6.0 Hz, 3H); ¹³C NMR (100 MHz, d₆-DMSO) δ158.1, 154.5, 148.3, 136.7, 131.8, 125.3, 124.7, 109.0, 107.9, 38.6, 31.0, 29.0, 26.1, 22.0, 21.3, 18.1, 13.9. HRMS (ESI) calcd for Cᵢ₇H₂₃NO₂([M+H]⁺) 274.1802; found: 274.1805.

5-Chloro-N-cyclooctylbenzofuran-2-carboxamide (24).

Purified by flash chromatography (100% CH₂Cl₂). Yield 79% (off-white solid). ¹H NMR (400 MHz, d₆-DMSO) δ8.57 (d, J=7.9 Hz, 1H), 7.86 (d, J=2.0 Hz, 1H), 7.69 (d, J=8.8 Hz, 1H), 7.52 (s, 1H), 7.47 (ddd, J=8.8, 2.1, 0.8 Hz, 1H), 4.04-3.98 (m, 1H), 1.75-1.65 (m, 6H), 1.60-1.45 (m, 8H); ¹³C NMR (100 MHz, d₆-DMSO) δ156.5, 152.6, 150.8, 128.8, 127.9, 126.5, 122.0, 113.4, 108.6, 49.1, 31.6 (2C), 26.7 (2C), 25.1, 23.5 (2C). HRMS (ESI) calcd for C₁₇H₂₀ClNO₂ ([M+H]⁺) 306.1255; found: 306.1252.

5-Chloro-N-(1-adamantyl)benzofuran-2-carboxamide (25).

Purified by re-crystallization from CH₃OH. Yield 84% (off-white solid). ¹H NMR (400 MHz, d₆-DMSO) δ7.84 (s, 1H), 7.78 (s, 1H), 7.68 (d, J=8.7 Hz, 1H), 7.51 (s, 1H), 7.45 (d, J=8.8 Hz, 1H), 2.07 (br s, 9H), 1.65 (m, 6H); ¹³C NMR (100 MHz, d₆-DMSO) δ157.0, 152.5, 151.0, 128.8, 127.9, 126.5, 121.9, 113.4, 108.5, 51.9, 40.7, 35.9, 28.8. HRMS (ESI) calcd for C₁₉H₂₀ClNO₂ ([M+H]⁺) 330.1255; found: 330.1264.

N-Cyclohexyl-1H-indole-2-carboxamide (26).

Yield 95% (white powder). ¹H NMR (400 MHz, d₆-DMSO) δ11.51 (s, 1H), 8.18 (d, J=8.0 Hz, 1H), 7.59 (d, J=7.9 Hz, 1H), 7.43 (d, J=8.2 Hz, 1H), 7.18-7.14 (m, 2H), 7.02 (t, J=7.8 Hz, 1H), 3.79 (br s, 1H), 1.85-1.59 (m, 5H), 1.38-1.27 (m, 4H), 1.15-1.13 (m, 1H); ¹³C NMR (100 MHz, d₆-DMSO) δ160.2, 136.4, 132.1, 127.1, 123.2, 121.4, 119.7, 112.3, 102.5, 48.0, 32.6, 25.3, 25.0. HRMS (ESI) calcd for C₁₅H₁₈N₂O ([M+H]⁺) 243.1491; found: 243.1498.

N-(3-Fluoro-4-methylphenyl)-1H-indole-2-carboxamide (27).

Yield 83% (white powder). ¹H NMR (400 MHz, d₆-DMSO) δ11.75 (s, 1H), 10.30 (s, 1H), 7.73 (d, J=12.0 Hz, 1H), 7.67 (d, J=8.0 Hz, 1H), 7.47 (d, J=8.0 Hz, 2H), 7.41 (s, 1H), 7.27-7.21 (m, 2H), 7.07 (t, J=8.0 Hz, 1H), 2.20 (s, 3H); ¹³C NMR (100 MHz, d₆-DMSO) δ161.5 (d, J=239 Hz), 159.8, 138.4 (d, J=11Hz), 136.9, 131.5 (d, J=6 Hz), 131.3, 127.0, 124.0, 121.9, 120.1, 118.9 (d, J=17 Hz), 115.7 (d, J=3 Hz), 112.5, 106.9 (d, J=27 Hz), 104.1, 13.7 (d, J=3 Hz). HRMS (ESI) calcd for C₁₆H₁₃FN₂O ([M+H]⁺) 269.1085; found: 269.1087.

N-Cycloheptyl-4,6-difluoro-1H-indole-2-carboxamide (28).

Yield 68% (white powder). ¹H NMR (400 MHz, d₆-DMSO) δ11.93, 8.33 (d, J=7.6 Hz, 1H), 7.29 (s, 1H), 7.03 (d, J=8.8 Hz), 6.87 (t, J=10.4 Hz, 1H), 3.98 (m, 1H), 1.89-1.85 (m, 2H), 1.65-1.42 (m, 10H); ¹³C NMR (100 MHz, d₆-DMSO) δ160.2 (d, J=236 Hz), 159.0, 157.0 (d, J=246 Hz), 137.5 (t, J=15.1 Hz), 133.0, 113.2 (d, J=22 Hz), 98.2, 95.3 (d, J=23 Hz), 94.7 (d, J=26 Hz), 50.1, 34.3, 27.9, 23.8. HRMS (ESI) calcd for C₁₆H₁₈F₂N₂O ([M+H]⁺) 293.1460; found: 293.1472

N-Cyclooctyl-4,6-difluoro-1H-indole-2-carboxamide (29).

Yield 69% (white powder). ¹H NMR (400 MHz, d₆-DMSO) δ11.93, 8.31 (d, J=8.0 Hz, 1H), 7.29 (s, 1H), 7.03 (d, J=9.6 Hz), 6.87 (t, J=10.4 Hz, 1H), 4.04-4.00 (m, 1H), 1.80-1.64 (m, 6H), 1.60-1.50 (m, 8H); ¹³C NMR (100 MHz, d₆-DMSO) δ160.2 (d, J=237 Hz), 159.0, 157.0 (d, J=247 Hz), 137.5 (t, J=14.9 Hz), 133.1, 113.1 (d, J=22 Hz), 98.2, 95.3 (d, J=23 Hz), 94.7 (d, J=26 Hz), 49.0, 31.5, 26.9, 25.0, 23.4. HRMS (ESI) calcd for C₁₇H₂₀F₂N₂O ([M+H]⁺) 307.1617; found: 307.1626.

N-(1-Adamantyl)-6-methoxy-1H-indole-2-carboxamide (30).

Purified by flash chromatography (EtOAc-hexane 1:3 to 3:2) followed by recrystallization from CH₃OH. Yield 77% (pale yellow powder). ¹H NMR (400 MHz, d₆-DMSO) δ11.23 (s, 1H), 7.46-7.42 (m, 2H), 7.08 (d, J=1.8 Hz, 1H), 6.87 (d, J=1.8 Hz, 1H), 6.67 (dd, J=8.7, 2.2 Hz, 1H), 3.76 (s, 3H), 2.09-2.06 (m, 9H), 1.67 (br s, 6H); ¹³C NMR (100 MHz, d₆-DMSO) δ160.5, 156.8, 137.2, 131.5, 122.1, 121.3, 110.7, 103.0, 94.1, 55.0, 51.4, 41.1, 36.1, 28.9. HRMS (ESI) calcd for C₂₀H₂₄N₂O₂ ([M+H]⁺) 325.1911; found: 325.1910.

N-(1-Adamantyl)-5-chloro-1H-indole-2-carboxamide (31).

Purified by flash chromatography (EtOAc-hexane 1:3 to 1:1) followed by re-crystallization from CH₃OH. Yield 40% (pale yellow powder). ¹H NMR (400 MHz, d₆-DMSO) δ11.63 (s, 1H), 7.65 (br s, 2H), 7.42 (d, J=8.7 Hz, 1H), 7.17-7.14 (m, 2H), 2.09-2.06 (m, 9H), 1.66 (br s, 6H); ¹³C NMR (100 MHz, d₆-DMSO) δ160.1, 134.6, 134.2, 128.1, 124.0, 123.1, 120.4, 113.7, 102.3, 51.7, 41.0, 36.0, 28.9. HRMS (ESI) calcd for C₁₉H₂₁ClN₂O ([M+H]⁺) 329.1415; found: 329.1399.

N-(1-Adamantyl)-6-hydroxy-1H-indole-2-carboxamide (32).

Compound 30 (0.73 mmol) was dissolved in anhydrous CH₂Cl₂ (7 mL) and cooled to 78° C. Subsequently BBr₃ (1.0 M solution in CH₂Cl₂, 4.4 mL, 6.0 equiv) was added dropwise and the reaction mixture was allowed to warm gradually to room temperature within 1 h. Stirring was continued at the same temperature an additional 3 h. The reaction was quenched with water and extracted with CH₂Cl₂ (2×50 mL). The combined organic phases were dried over Na₂SO₄, filtered and concentrated under reduced pressure. The crude material was purified by column chromatography (EtOAc-hexane 1:3 to 1:1) followed by preparative HPLC. Yield 52% (white powder). ¹H NMR (400 MHz, d₆-DMSO) δ11.00 (s, 1H), 9.11 (s, 1H), 7.35-7.33 (m, 2H), 7.01 (d, J=1.5 Hz, 1H), 6.76 (d, J=1.5 Hz, 1H), 6.55 (dd, J=8.6, 2.1 Hz, 1H), 2.07 (br s, 9H), 1.66 (br s, 6H); ¹³C NMR (100 MHz, d₆-DMSO) δ160.6, 154.6, 137.6, 131.0, 121.9, 120.6, 111.0, 103.1, 96.4, 51.4, 41.1, 36.1, 28.9. HRMS (ESI) calcd for C₁₉H₂₂N₂O₂([M+H]⁺) 311.1754; found: 311.1767.

N-Cyclooctyl-6-methoxy-1H-pyrrolo[3,2-c]pyridine-2-carboxamide (33).

Yield 80% (white solid). ¹H NMR (400 MHz, d₆-DMSO) δ11.67 (s, 1H), 8.39 (br s, 2H), 7.04 (s, 1H), 6.91 (s, 1H), 4.09-3.99 (m, 1H), 3.83 (s, 3H), 1.80-1.48 (m, 14H); ¹³C NMR (400 MHz, d₆-DMSO) δ159.3, 157.4, 137.3, 135.7, 131.7, 130.7, 100.4, 97.5, 53.6, 49.2, 31.6, 26.9, 25.1, 23.5. HRMS (ESI) calcd for C₁₇H₂₃N₃O₂([M+H]⁺) 302.1863; found: 302.1874.

N-(1-Adamantyl)-6-methoxy-1H-pyrrolo[3,2-c]pyridine-2-carboxamide (34).

Yield 70% (white solid).¹H NMR (400 MHz, d₆-DMSO) δ11.60 (s, 1H), 8.39 (s, 1H), 7.75 (s, 1H), 7.04 (s, 1H), 6.90 (s, 1H), 3.83 (s, 3H), 2.09 (br s, 9H), 1.67 (s, 6H); ¹³C NMR (100 MHz, d₆-DMSO) δ159.9, 157.4, 137.9, 135.7, 131.6, 130.6, 100.7, 97.5, 53.6, 51.9, 41.0, 36.1, 28.9. HRMS (ESI) calcd for C₁₉H₂₃N₃O₂ ([M+H]⁺) 326.1863; found: 326.1867.

N-Cycloheptyl-4,6-bis(trifluoromethyl)-1H-indole-2-carboxamide (35).

Yield 54% (white powder). ¹H NMR (400 MHz, d₆-DMSO) δ12.59 (s, 1H), 8.70 (d, J=8.0 Hz, 1H), 8.03 (s, 1H), 7.66 (s, 1H), 7.54 (s, 1H), 4.04 (m, 1H), 1.93-1.87 (m, 2H), 1.71-1.55 (m, 10H); ¹³C NMR (100 MHz, d₆-DMSO) δ158.5, 137.0, 135.7, 125.6 (d, J=21 Hz), 125.3, 122.9 (d, J=21 Hz), 122.8 (q, J=32 Hz), 122.0 (q, J=33 Hz), 114.1, 113.4, 100.4, 50.3, 34.3, 27.9, 23.8. HRMS (ESI) calcd for C₁₈Hᵢ₈F₆N₂O ([M+H]⁺) 393.1396; found: 393.1386.

N-Cyclooctyl-4,6-bis(trifluoromethyl)-1H-indole-2-carboxamide (36).

Yield 61% (white powder). ¹H NMR (400 MHz, d₆-DMSO) δ12.58 (s, 1H), 8.67 (d, J=8.0 Hz, 1H), 8.03 (s, 1H), 7.66 (s, 1H), 7.55 (s, 1H), 4.07 (m, 1H), 1.82-1.51 (m, 14H); ¹³C NMR (100 MHz, d₆-DMSO) δ158.5, 137.0, 135.7, 125.7 (d, J=20 Hz), 125.3, 123.0 (d, J=21 Hz), 122.8 (q, J=32 Hz), 122.0 (q, J=33 Hz), 114.0, 113.4, 100.4, 49.3, 31.4, 26.8, 25.0, 23.4. HRMS (ESI) calcd for C₁₉H₂₀F₆N₂O ([M+H]⁺) 407.1553; found:407.1562.

N-Cycloheptyl-4,6-dimethyl-1H-benzo[d]imidazole-2-carboxamide (37).

Yield 36% (white powder). ¹H NMR (400 MHz, d₆-DMSO) δ8.83 (s, 1H), 7.24 (s, 1H), 7.00 (s, 1H), 4.04-4.00 (m, 1H), 2.55 (s, 3H), 2.40 (s, 3H), 1.91-1.87 (m, 2H), 1.72-1.42 (m, 10H); ¹³C NMR (100 MHz, d₆-DMSO) δ153.7, 142.3, 136.0, 132.8, 131.4, 127.9, 125.4, 111.3, 51.0, 33.8, 27.9, 23.6, 21.2, 16.7. HRMS (ESI) calcd for C₁₇H₂₃N₃O ([M+H]⁺) 286.1914; found: 286.1921.

N-Cyclooctyl-4,6-dimethyl-1H-benzo[d]imidazole-2-carboxamide (38).

Yield 51% (white powder). ¹H NMR (400 MHz, d₆-DMSO) δ9.33 (s, 1H), 7.29 (s, 1H), 7.10 (s, 1H), 4.12-4.04 (m, 1H), 2.58 (s, 3H), 2.42 (s, 3H), 1.81-1.72 (m, 6H), 1.63-1.54 (m, 8H); ¹³C NMR (100 MHz, d₆-DMSO) δ154.3, 142.8, 135.4, 133.6, 132.5, 127.4, 125.5, 111.5, 49.9, 31.0, 26.8, 24.9, 23.3, 21.2, 16.7. HRMS (ESI) calcd for C₁₈H₂₅N₃O ([M+H]⁺) 300.2070; found: 300.2080.

N-(1-Adamantyl)-4,6-dimethyl-1H-benzo[d]imidazole-2-carboxamide (39).

Yield 40% (white powder). ¹H NMR (400 MHz, d₆-DMSO) δ7.94 (s, 1H), 7.23 (s, 1H), 6.98 (s, 1H), 2.52 (s, 3H), 2.39 (s, 3H), 2.11-2.09 (br s, 9H), 1.68 (s, 6H); ¹³C NMR (100 MHz, d₆-DMSO) δ156.3, 144.3, 135.6, 134.9, 134.0, 126.2, 126.0, 111.7, 52.0, 40.7, 35.8, 28.9, 21.2, 16.6. HRMS (ESI) calcd for C₂₀H₂₅N₃O ([M+H]⁺) 324.2070; found: 324.2077.

N-(2-Adamantyl)-4,6-dimethyl-1H-benzo[d]imidazole-2-carboxamide (40). Yield 44% (white powder). ¹H NMR (400 MHz, d₆-DMSO) δ8.12 (d, J=7.2 Hz, 1H), 7.23 (s, 1H), 6.96 (s, 1H), 4.11 (d, J=7.6 Hz, 1H), 2.53 (s, 3H), 2.39 (s, 3H), 2.02 (s, 2H), 1.99 (d, J=13.2 Hz, 2H), 1.86 (br s, 6H), 1.74 (s, 2H), 1.65 (d, J=12.4 Hz, 2H); ¹³C NMR (100 MHz, d₆-DMSO) δ157.2, 144.2, 136.6, 135.9, 133.5, 126.2, 125.8, 112.1, 53.4, 36.9, 36.5, 31.2, 26.6, 21.2, 16.6. HRMS (ESI) calcd for C₂₀H₂₅N₃O ([M+H]⁺) 324.2070; found: 324.2076.
N-Cycloheptyl-4,6-dimethyl-1H-indole-3-carboxamide (41).

Yield 44% (off-white powder). ¹H NMR (400 MHz, d₆-DMSO) δ11.17 (s, 1H), 7.77 (d, J=8.0 Hz, 1H), 7.52 (d, J=2.4 Hz, 1H), 7.00 (s, 1H), 6.65 (s, 1H), 3.94 (m, 1H), 2.53 (s, 3H), 2.33 (s, 3H), 1.90-1.86 (m, 2H), 1.68-1.56 (m, 10H); ¹³C NMR (100 MHz, d₆-DMSO) δ164.5, 136.8, 130.7, 130.1, 126.2, 123.2, 122.3, 113.6, 109.0, 50.0, 34.4, 27.8, 24.0, 21.1, 21.0. HRMS (ESI) calcd for C₁₈H₂₄N₂O ([M+H]⁺) 285.1961; found: 285.1973.

N-Cyclooctyl-4,6-dimethyl-1H-indole-3-carboxamide (42).

Yield 34% (off-white powder). ¹H NMR (400 MHz, d₆-DMSO) δ11.17 (s, 1H), 7.76 (d, J=8.0 Hz, 1H), 7.52 (d, J=2.4 Hz, 1H), 6.99 (s, 1H), 6.65 (s, 1H), 3.96 (m, 1H), 2.53 (s, 3H), 2.33 (s, 3H), 1.77-1.49 (m, 14H); ¹³C NMR (100 MHz, d₆-DMSO) δ164.4, 136.7, 130.7, 130.1, 126.2, 123.2, 122.3, 113.7, 109.0, 48.7, 31.7, 26.9, 25.1, 23.6, 21.1, 20.9. HRMS (ESI) calcd for C₁₉H₂₆N₂O ([M+H]⁺) 299.2118; found: 299.2119.

N-(1-Adamantyl)-4,6-dimethyl-1H-indole-3-carboxamide (43).

Yield 38% (off-white powder). ¹H NMR (400 MHz, d₆-DMSO) δ11.11 (s, 1H), 7.47 (d, J=2.8 Hz, 1H), 7.26 (s, 1H), 6.98 (s, 1H), 6.64 (s, 1H), 2.52 (s, 3H), 2.33 (s, 3H), 2.07-2.05 (m, 9H), 1.66 (s, 6H); ¹³C NMR (100 MHz, d₆-DMSO) δ165.5, 136.6, 130.6, 130.0, 125.9, 123.1, 122.3, 114.7, 109.0, 51.0, 41.1, 36.2, 28.9, 21.1, 20.8. HRMS (ESI) calcd for C₂₁H₂₆N₂O ([M+H]⁺), 323.2118; found: 323.2109

N-(2-Adamantyl)-4,6-dimethyl-1H-indole-3-carboxamide (44).

Yield 42% (off-white powder). ¹H NMR (400 MHz, d₆-DMSO) δ11.21 (s, 1H), 7.65 (d, J=6.8 Hz, 1H), 7.60 (d, J=2.4 Hz, 1H), 7.01 (s, 1H), 6.65 (s, 1H), 4.03 (m, 1H), 2.52 (s, 3H), 2.34 (s, 3H), 2.14 (d, J=12.8 Hz, 2H), 1.96 (s, 2H), 1.85-1.79 (m, 6H), 1.72 (s, 2H), 1.53 (d, J=12.8 Hz, 2H); ¹³C NMR (100 MHz, d₆-DMSO) δ165.3, 136.8, 130.7, 130.1, 126.7, 123.2, 122.4, 113.4, 109.1, 53.4, 37.3, 37.0, 31.4, 31.1, 26.9, 21.1, 20.8. HRMS (ESI) calcd for C₂₁H₂₆N₂O ([M+H]⁺) 323.2118; found: 323.2110.

N-Cyclooctyl-4,6-dichloro-1H-indole-2-carboxamide (70).

Yield 69% (white powder). ¹H NMR (400 MHz, d₆-DMSO) δ12.02 (s, NH), 8.46 (d, J=8.0 Hz, 1H), 7.41 (s, 1H), 7.33 (s, 1H), 7.21 (s, 1H), 4.06-4.01 (m, 1H), 1.81-1.50 (m, 14H); ¹³C NMR (100 MHz, d₆-DMSO) δ158.9, 136.7, 133.8, 127.5, 126.2, 124.8, 119.3, 111.0, 100.6, 49.1, 31.4, 26.9, 25.0, 23.4.

### Biology.

MIC was determined by using MABA as reported previously.^{11,12} Cytotoxicity was evaluated on Vero cells also by using MABA format.¹¹ Oral bioavalability was analyzed by using serum inhibition titration assay.¹³ Briefly, compounds were ground to homogenate suspension in 0.5% carboxymethyl cellulose. Six-week old female BALB/c mice were single-dosed at 300 or 100 mg/kg by oral gavage. Isoniazid at 10 mg/kg was used as positive control and 0.5% carboxymethyl cellulose treatment was used as vehicle control. At 15, 30 and 60 min after administration, cardiac blood was collected and serum was separated. Two-fold serial titration was carried out using 96-well plates, 10⁴ colony forming units of M. tuberculosis H37Rv were added to testing wells. Plates were then incubated and processed as regular MABA.

### Bacterial Strains.

Wild type M. tuberculosis H37Rv lab strain was obtained from the Johns Hopkins Center for Tuberculosis Research laboratory stocks. The KwaZulu-Natal clinical isolates used in this study were a kind gift from Dr. William R. Jacobs, Jr., at the Albert Einstein College of Medicine.

### MIC and MBC Assays.

MIC was determined using microplate alamar blue assay^{11,12.} Plates were then read using a fluorescence microplate reader at 544 ex/590 em. Percentage inhibition was calculated based on the relative fluorescence units and the minimum concentration that resulted in at least 90% inhibition was identified as MIC. For this assay, 7H9 broth without Tween-80 was used as the assay media.

For MIC and MBC determination using tube-broth dilution methods, compounds 3, 11 and 12 were 2-fold serially diluted at a volume of 2.5 mL in 7H9 without Tween-80. Mid-log phase H37Rv culture was diluted, and 0.1 mL of the diluted culture containing 10⁵ CFUs was added to each of the assay tubes. Media control, positive control (isoniazid) and growth control (no compound) were included. Tubes were incubated at 37° C. At day 7 and day 14, pellet formation was observed and recorded and the minimum concentration that prevented pellet formation was identified as MIC. The end point CFUs per tube for the treatment was determined on the tubes that did not show pellet on Day 14. The minimum concentration that killed 99% of the inoculum was identified as the MBC.

### Kill Kinetic Assay.

M. tuberculosis H37Rv culture was diluted to an OD₆₀₀ of 0.001 and then divided to five of 10 mL aliquots and supplemented with a final concentration of 0.016 μg/mL (4× MIC) or 0.064 μg/mL (16× MIC) of compound 12, or 0.125 μg/mL (4× MIC) or 0.5 μg/mL (16× MIC) of compound 11. At day 0, 1, 3, and 5, cultures were diluted and plated. CFUs per mL were enumerated after 4 weeks of incubation.

### Cytotoxicity Assay.

Vero cell linage (ATCC CCL-81) was grown in Dulbecco's Modified Eagle Medium (DMEM) containing 10% fetal bovine serum (FBS). Flat-bottomed 96-well plate was seeded with 4×10⁴ cells. The plate was incubated at 37° C. with 5% CO₂ for 16 h. For compound preparation, 2-fold serial dilution was made using a deep-well block using DMEM containing 5% FBS with a volume of 200 μL. Culture media was replaced with 160 μL of the compound-containing media, with 100% DMSO as positive (100% kill) control and media only as blank (100% viability) control. The plate was incubated for 72 h and then washed twice with PBS before adding 100 μL of DMEM with 5% FBS medium freshly mixed with 10% alamar blue. The plate was incubated for 2 h and then immediately read with a fluorescence microplate reader at 544Ex/590Em. The minimum concentration that killed at least 50% of the cells was identified as IC₅₀.

### Selection of Indoleamide-Resistant Mutant.

To select for resistance, 7H10 agar plates containing 2×, 4×, 8× and 16× MIC of compound 11 were prepared. Late log phase M. tuberculosis H37Rv culture (0D₆₀₀ approximately 1.0) was spread on these plates and incubated at 37° C. for 4 weeks. Colonies were recovered and propagated in 7H9 broth containing correspondent level of the compound.

Deep sequencing and Target Identification.

Genomic DNA was isolated from both the parental wild type (H37Rv) and the resistant mutant (IAR2) strain by using the lysozyme and cetyltrimethylammonium bromide in glucose-tris-EDTA buffer methods. 5 μg DNA was subjected to Covaris S2 DNA shearing system to prepare DNA fragments. The library was prepared and enriched by using the Ion OneTouch and Ion OneTouch Template Kit systems. Enriched template-positive Ion Sphere Particles was sequenced using the Ion Torrent Personal Genome Machine following the Ion 316 Chip protocol and the Ion Sequencing Kit User Guide v2.0 (Life Technologies). After on-machine filtering, all reads were tempted to be aligned to the published M. tuberculosis H37Rv sequence° by using the Burrows-Wheeler Aligner algorithms¹⁴. SNPs were analyzed and called by the GATK package.

### Mouse Aerosol Infection and Monotherapy Model.

Four-to-six-week-old female BALB/c mice were aerosol-infected with M. tuberculosis H37Ry. From 14 days after infection, group of five mice were treated with 33.3, 100 and 300 mg/kg of compound 3 by oral gavage, daily (5 days per week). Isoniazid at 10 mg/kg was administered as positive control. Infected but untreated mice were negative control. At day—13, 0, 7, 14, and 28 from treatment start, 5 mice from each treatment were sacrificed and the lungs removed. The lungs were bead-beaten to homogenate, diluted and plated on 7H11 selective agar plates. All animal procedures were approved by the Institutional Animal Care and Use Committee of the Johns Hopkins University School of Medicine.

### In Vivo Pharmacokinetic Evaluation.

Female BALB/c mice (20 g each, Charles River Laboratories) were given a single dose of compound 12 at 100 mg/kg by oral gavage in a volume of 0.2 mL. At 0.125, 0.25, 0.5, 1, 2, 4, 8 and 24 h after compound administration, animals (n=3 per time point) were euthanized and cardiac blood (-0.7 mL) was collected. Mouse lungs were removed, weighed and stored at −80° C. Plasma was separated by centrifugation at 12,000 x g for 20 min at 4° C. and stored at −80° C. Mouse lungs were homogenized by bead-beating in 0.5 mL of liquid chromatography/mass spectrometry (LC/MS) water and supernatants were recovered by centrifugation at 4° C. for 20 min. Concentrations of compound 12 in plasma and lung homogenate supernatants were analyzed with LC-tandem MS (LC-MS/MS, AB SCIEX QTRAP 5500 system) with compound 2 as internal standard. MS detection of mass transitions 299.01/146.1 and 299.01/131.1 was carried out. Concentration calculation was done with MultiQuant Software (Version 2.1, AB SCIEX). The pharmacokinetic profile of the test compound was analyzed from plasma and lung concentration-time data after oral administration. The peak concentration (Cₘₐₓ), the time of peak (Tₘₐₓ), and the area under the concentration curve from time 0 to 24 h (AUC₀₋₂₄) were calculated by using GraphPad Prism 4.

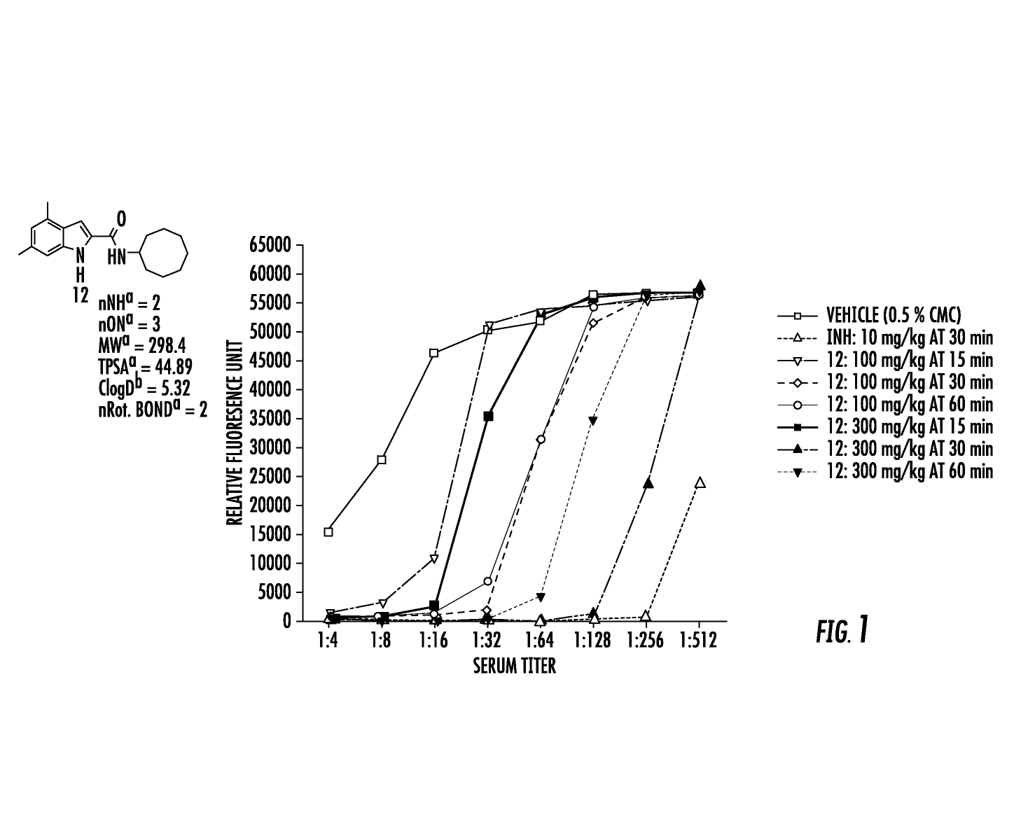

Indole-2-carboxamides 11-14 were evaluated in the serum inhibition titration assay.¹³ Briefly, each compound was administered at 100 and 300 mg/kg to BALB/c mice by oral gavage using carboxymethyl cellulose as vehicle, after which blood samples were collected at 15, 30 and 60 minutes. The sera were separated and prepared in 2-fold dilutions and incubated with a bacterial suspension for 7 days. Bacterial growth was measured using MABA. The results are shown in FIG. 1.

TABLE 1
Antitubercular activity of compounds 3-18 against the M. tuberculosis
strain H37Rv.

| | | | 3-17 |
|---|---|---|---|
| 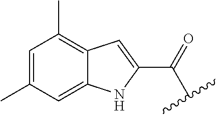 [SMILES: *C(=O)c1cc2c(C)cc(C)cc2[nH]1] | | | |
| | | | 18 |
| 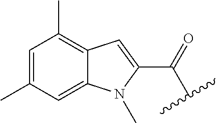 [SMILES: *C(=O)c1cc2c(C)cc(C)cc2n1C] | | | |
| | | MIC^{a} | IC₅₀^{b} |
| Compd | R | (μM) | (μM) |
| 3 | 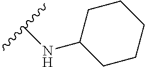 [SMILES: *NC1CCCCC1] | 0.93 | >200 |
| 4 | 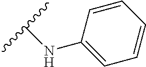 [SMILES: *Nc1ccccc1] | 3.8 | >200 |
| 5 | 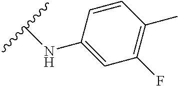 [SMILES: *Nc1ccc(C)c(F)c1] | 1.7 | >200 |
| 6 | 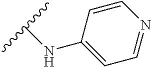 [SMILES: *Nc1ccncc1] | 240 | NT^{c} |
| 7 | 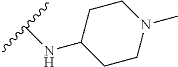 [SMILES: *NC1CCN(C)CC1] | 448 | NT |
| 8 | 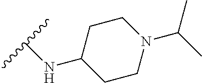 [SMILES: *NC1CCN(C(C)C)CC1] | 204 | NT |
| 9 | 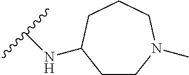 [SMILES: *NC1CCCN(C)CC1] | 428 | NT |
| 10 | 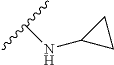 [SMILES: *NC1CC1] | 561 | NT |
| 11 | 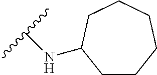 [SMILES: *NC1CCCCCC1] | 0.055 | >200 |
| 12 | 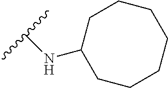 [SMILES: *NC1CCCCCCC1] | 0.013 | 54 |
| 13 | 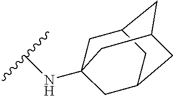 [SMILES: *NC12CC3CC(CC(C3)C1)C2] | 0.012 | >200 |
| 14 | 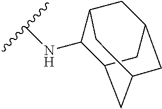 [SMILES: *NC1C2CC3CC(C2)CC1C3] | 0.012 | >200 |
| 15 | 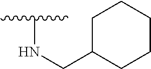 [SMILES: *NCC1CCCCC1] | 0.88 | >200 |
| 16 | 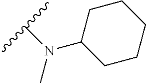 [SMILES: *N(C)C1CCCCC1] | 450 | NT |
| 17 | 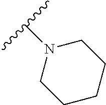 [SMILES: *N1CCCCC1] | >499 | NT |
| 18 | 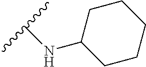 [SMILES: *NC1CCCCC1] | 450 | NT |
| INH^{d} | | 0.29 | NT |

^{a}The lowest concentration of drug leading to at least a 90% reduction of bacterial growth signal by the microplate Alamar Blue assay (MABA). MIC values are reported as an average of three individual measurements;
^{b}cytotoxicity against Vero cells;
^{c}NT = not tested;
^{d}INH = Isoniazid.

TABLE 2
Antitubercular activity of compounds 19-40 against M. tuberculosis
strain H37Rv.

| | |
|---|---|
| 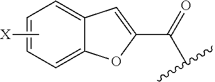 | 19-25 |
| 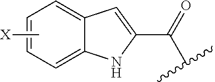 | 26-32, 35-36 |
| 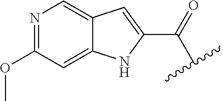 [SMILES: *C(=O)c1cc2cnc(OC)cc2[nH]1] | 33-34 |
| 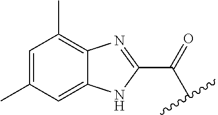 [SMILES: *C(=O)c1nc2c(C)cc(C)cc2[nH]1] | 37-40 |

| | | | MIC^{a} |
|---|---|---|---|
| Compd | X | R | (μM) |
| 19 | 4,6- dimethyl | 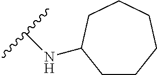 [SMILES: *NC1CCCCCC1] | 56 |
| 20 | 4,6- dimethyl | 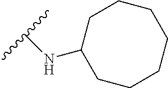 [SMILES: *NC1CCCCCCC1] | 27 |
| 21 | 4,6- dimethyl | 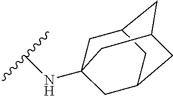 [SMILES: *NC12CC3CC(CC(C3)C1)C2] | 3.1 |
| 22 | 4,6- dimethyl | 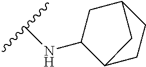 [SMILES: *NC1CC2CCC1C2] | 113 |
| 23 | 4,6- dimethyl | 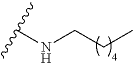 | 59 |
| 24 | 5-Cl | 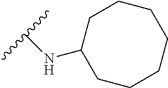 [SMILES: *NC1CCCCCCC1] | 26 |
| 25 | 5-Cl | 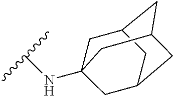 [SMILES: *NC12CC3CC(CC(C3)C1)C2] | ≧388 |
| 26 | H | 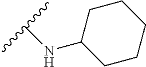 [SMILES: *NC1CCCCC1] | >528 |
| 27 | H | 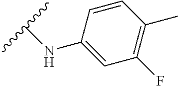 [SMILES: *Nc1ccc(C)c(F)c1] | 477 |
| 28 | 4,6- difluoro | 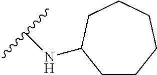 [SMILES: *NC1CCCCCC1] | 0.86 |
| 29 | 4,6- difluoro | 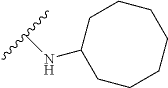 [SMILES: *NC1CCCCCCC1] | 0.10 |
| 30 | 6-OCH₃ | 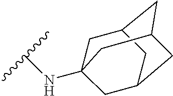 [SMILES: *NC12CC3CC(CC(C3)C1)C2] | 0.77 |
| 31 | 5-Cl | 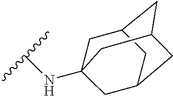 [SMILES: *NC12CC3CC(CC(C3)C1)C2] | 0.38 |
| 32 | 6-OH | 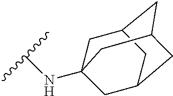 [SMILES: *NC12CC3CC(CC(C3)C1)C2] | 13 |
| 33 | — | 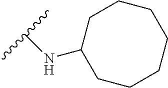 [SMILES: *NC1CCCCCCC1] | 6.6 |
| 34 | — | 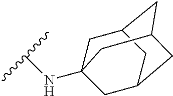 [SMILES: *NC12CC3CC(CC(C3)C1)C2] | 1.5 |
| 35 | 4,6- bis(CF₃) | 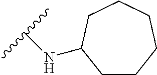 [SMILES: *NC1CCCCCC1] | 0.64 |
| 36 | 4,6- bis(CF₃) | 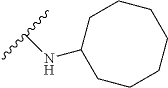 [SMILES: *NC1CCCCCCC1] | 0.04 |
| 37 | — | 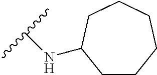 [SMILES: *NC1CCCCCC1] | >224 |
| 38 | — | 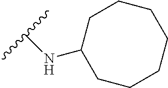 [SMILES: *NC1CCCCCCC1] | 1.7 |
| 39 | — | 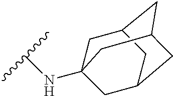 [SMILES: *NC12CC3CC(CC(C3)C1)C2] | 0.39 |
| 40 | — | 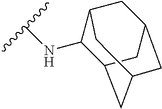 [SMILES: *NC1C2CC3CC(C2)CC1C3] | 1.5 |

^{a}The lowest concentration of drug leading to at least a 90% reduction of bacterial growth signal by microplate Alamar Blue assay (MABA). MIC values are reported as an average of three individual measurements.

TABLE 3
Antitubercular activity of compound 3, 11 and 12 against
susceptible, MDR and XDR strains of M. tuberculosis.

| | | | | | |
|---|---|---|---|---|---|
| | V4207 | TF274 | R506 | KZN494 | V2475 |
| Compd | (DS)^{a} | (XDR)^{b} | (XDR)^{b} | (MDR)^{c} | (MDR)^{c} |

MIC^{d} (μM)

| | | | | | |
|---|---|---|---|---|---|
| 3 | 0.93 | 0.46 | 0.46 | 3.7 | 0.93-1.9 |
| 11 | 0.11 | 0.055 | 0.055 | 0.11 | 0.11 |
| 12 | 0.026 | 0.026 | 0.0067 | ^{e}NT | ^{e}NT |

^{a}Drug susceptible strain of M. tuberculosis;
^{b}extensively drug resistant strain of M. tuberculosis;
^{c}multi-drug resistant strain of M. tuberculosis;
^{d}the lowest concentration of drug leading to at least a 90% reduction of bacterial growth signal by microplate Alamar Blue assay (MABA); reported MIC values are an average of three individual measurements;
^{e}NT = not tested.

Selected compounds 3, 11 and 12 were tested for their ability to inhibit the growth of the acquired clinical MDR-TB (KZN494 and V2475) and XDR-TB (TF274 and R506) strains from KwaZulu-Natal, South Africa (Table 3).¹⁵ To our delight, these indole-2-carboxamides maintained similar excellent activities against the susceptible M. tuberculosis strain H₃₇Rv in all the tested drug-resistant strains.

TABLE 4
Summary statistics of whole genome sequencing.

| | | | | | | | Average | | | |
|---|---|---|---|---|---|---|---|---|---|---|
| | | Total | | | | Percent | Coverage | | | |
| Sample | Chip | Bases | AQ17 | AQ20 | Perfect | Coverage | Depth | SNPs | Indels | Gaps |
| H37Rv | 314 | 50.41 | 40.73 | 36.97 | 32.48 | 98% | 11.43X | 81 | 41 | 687 |
| | 316 | 131.43 | 105.54 | 94.00 | 81.71 | 96% | 29.80X | 79 | 10 | 1831 |
| IAR2 | 314 | 38.52 | 33.81 | 31.30 | 28.73 | 99% | 8.73X | 82 | 26 | 559 |
| | 316 | 154.07 | 126.29 | 113.06 | 103.64 | 98% | 34.94X | 89 | 14 | 1236 |

Sequencing was performed using the Ion Torrent Personal Genome Machine platform. Each genome was sequenced twice. The reference sequence for the annotation of both strains is the published M. tuberculosis H37Rv genome, NCBI Reference Sequence NC_000962¹⁴. Chip, Ion Torrent semiconductor chip type; Total Bases, total mega bases of DNA sequenced; AQ17, mega bases of DNA with one mismatch in the first 50 bases relative to the reference strain; AQ20, mega bases of DNA with one mismatch in the first 100 bases relative to the reference strain; Perfect, mega bases of DNA with perfect alignment relative to the reference strain; SNPs, Single nucleotide polymorphisms relative to the published reference genome Indels, Insertions/deletions relative to the published reference genome; Gaps, Gaps in the complete sequence relative to the published reference genome.

TABLE 5
Single nucleotide polymorphisms identified in
the Mycobacterium tuberculosis IAR2 isolate.

| SNP Description | SNP/Coverage | Locus Tag | Gene Name | SNP Class | AA Change |
|---|---|---|---|---|---|
| A 246,457 T | 14/14 | Rv0206c | mmpL3 | Missense | S 288 T |
| A 340,613 G | 2/2 | Rv0280 | PPE3 | Missense | D 417 G |
| C 1,655,844 T | 2/2 | Rv1468c | PE_PGRS29 | Missense | S 293 N |

The reference sequence for the annotation of both strains is the published M. tuberculosis H37Rv genome, NCBI Reference Sequence NC_000962 ¹⁴. In addition to the SNP in mmpL3, two other SNPs were identified, but only with 2 sequence reads each.
SNP Description, the position of the SNP relative to the reference genome with the reference base to the left of the position and the observed base to the right; SNP/Coverage, the number of times the described SNP was observed over the total number of transcripts covering that allele; AA amino acid.

TABLE 6
MIC of indoleamides and three additional compounds reported
to target the MmpL3 mycolic acid transporter.

| | MIC (μg/mL) | MIC (μg/mL) | Fold change in |
|---|---|---|---|
| Compound | for H37Rv | for IAR2 | MIC for IAR2 |
| compound 3 | 0.125-0.25 | ≧128 | ≧(512-1024) |
| compound 11 | 0.0156-0.0313 | 1 | 32-64 |
| compound 12 | 0.0039 | 0.25 | 64 |
| AU1235 | 0.0313-0.0625 | >64 | >(1024-2048) |
| SQ109 | 0.25 | 4 | 16 |
| BM212 | 2 | 4 | 2 |
| Isoniazid | 0.04 | 0.04 | 0 |
| Rifampin | 0.125 | 0.03125 | 0.25 |
| Ethambutol | 1 | 1 | 0 |
| Levofloxacin | 0.25 | 0.25 | 0 |
| Moxifloxacin | 0.0625-0.125 | 0.0625-0.125 | 0 |
| Kanamycin | 2 | 2 | 0 |
| Capreomycin | 1 | 1 | 0 |
| Amikacin | 1 | 1 | 0 |

MIC, minimum inhibitory concentration

TABLE 7
Bacterial burden in mouse lungs.

| | Mean lung CFU counts (standard deviation) at the following time points: | | | | |
|---|---|---|---|---|---|
| Treatment | Day −14 | Day 0 | Day 7 | Day 14 | Day 28 |
| Untreated | 2.971 (0.039) | 6.545 (0.046) | 7.136 (0.285) | 6.936 (0.366) | 7.300 (0.025) |
| Isoniazid | — | — | 5.508 (0.124) | 5.266 (0.089) | 4.561 (0.088) |
| (10 mg/kg) | | | | | |
| Compound 12 | — | — | 7.184 (0.244) | 7.001 (0.206) | 6.919 (0.112) |
| (33.3 mg/kg) | | | | | |
| Compound 12 | — | — | 6.902 (0.243) | 7.122 (0.148) | 6.803 (0.068) |
| (100 mg/kg) | | | | | |
| Compound 12 | — | — | 6.768 (0.329) | 6.981 (0.305) | 6.746 (0.157) |
| (300 mg/kg) | | | | | |

Mean colony forming unit (CFU) counts from the lungs of M. tuberculosis-infected mice before and during treatment with compound 12. Five mice per group were sacrificed at each time point, except for untreated control at Day 28, which was four mice because of an accidental death prematurely. Day -14 represents the day after infection, and day 0 represents the day of treatment initiation. Drugs were administered daily (5 days per week) by oral gavage.

TABLE 8
In vivo pharmacokinetic parameters of
compound 12 in female BALB/c mice.

| | | |
|---|---|---|
| Cₘₐₓ (SEM) | Tₘₐₓ | AUC₀₋₂₄ |

| | | | | | |
|---|---|---|---|---|---|
| Plasma | 0.49 (0.271) | μg/mL | 2.00 h | 3.71 | mg · h/L |
| Lung | 2.47 (1.507) | μg/g | 4.00 h | 31.40 | mg · h/kg |

A single 100 mg/kg dose of compound 12 was administered to 24 mice (3 per time point). Plasma and lung concentration of compound 12 was determined by liquid chromatography-tandem mass spectrometry. Cₘₐₓ, maximum concentration; Tₘₐₓ, time to maximum concentration, AUC₀₋₂₄, area under the concentration curve during the first 24 hours post-administration; SEM, standard error of the mean.

Whole-cell phenotypic high-throughput screening is a powerful tool for evaluation of the antimicrobial activity of compounds in large chemical libraries. Indeed, such high-throughput compound screening with the proxy nonpathogenic organism M. smegmatis identified the diarylquinoline precursor to bedaquiline, which was subsequently optimized for activity against M. tuberculosis¹⁶. This method has been adapted for direct utility with M. tuberculosis and has led to the identification of a number of promising lead compounds¹⁷. A recent phenotypic screening of a library of 6,800 compounds identified several chemotypes with anti-M. tuberculosis activity^{11,12,18,19}. We synthesized and preliminarily characterized one molecular class, indoleamides, which was active against both drug-susceptible and drug-resistant M. tuberculosis²⁰. Here we further characterize three lead compounds from this class both in vitro and in vivo. Our work indicates that these compounds target the mycobacterial membrane protein, large-3 (MmpL3), a mycolic acid transporter, and that the indoleamides are orally bioavailable and effective in vivo in a mouse model of TB, indicating promising translational potential.

TABLE 9
Hit 1a analogs tested for anti-M.tb. (H37Rv strain) activity (IC₅₀ (MABA), MIC₉₀ (BD),
MBC) and cytotoxicity to Vero cells.

| | | | | | | | | | | 1t-1w |
|---|---|---|---|---|---|---|---|---|---|---|
| 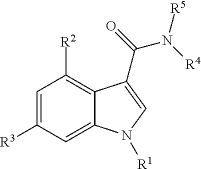 | | | | | | | | | | |
| | | | | | | | | | | 1a-1s |
| 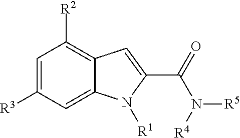 | | | | | | | | | | |
| | | | | | | | | | IC₅₀ | Selectivity |
| Comp 1 | R¹ | R² | R³ | R⁴ | R⁵ | MIC_{MABA} | MIC_{BD} | MBC_{BD} | Vero Cells | Index |
| A | H | CH₃ | CH₃ | H | c-Hexyl | 0.125 | 0.25 | 0.25 | >64 | >256 |
| B | H | CH₃ | CH₃ | H | Ph | 1 | 1 | 8 | >64 | |
| C | H | CH₃ | CH₃ | H | 3-F-4-Me—Ph | 0.25 | 0.5 | 8 | >64 | |
| D | H | CH₃ | CH₃ | H | c-Propyl | 64 | 128 | NT | NT | |
| E | H | H | H | H | c-Hexyl | 128 | >128 | NT | NT | |
| F | H | H | H | H | 3-F-4-Me—Ph | 128 | 128 | NT | NT | |
| G | H | CH₃ | CH₃ | H | c-Heptyl | 0.0156 | 0.0156 | 0.0312 | >64 | >2048 |
| H | H | CH₃ | CH₃ | H | c-Octyl | 0.0039 | NT | NT | 16 | 4000 |
| I | H | CH₃ | CH₃ | H | 1- | 0.0019 | NT | NT | >64 | |
| | | | | | Adamantyl | | | | | |
| J | H | CH₃ | CH₃ | H | 2- | 0.0039 | NT | NT | >64 | |
| | | | | | Adamantyl | | | | | |
| K | H | CH₃ | CH₃ | H | 4-Pyridyl | 32 | 64 | NT | NT | |
| L | H | CH₃ | CH₃ | H | —CH₂-c- | 0.25 | 0.25 | 0.25 | >64 | |
| | | | | | Hexyl | | | | | |
| M | CH₃ | CH₃ | CH₃ | H | c-Hexyl | 128 | 128 | NT | NT | |
| N | H | CH₃ | CH₃ | CH₃ | c-Hexyl | 32 | 128 | NT | NT | |
| O | H | CH₃ | CH₃ | H | 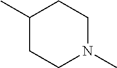 | 128 | NT | NT | NT | |
| P | H | CH₃ | CH₃ | H | 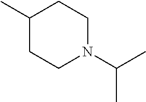 | 64 | NT | NT | NT | |
| Q | H | CH₃ | CH₃ | H | 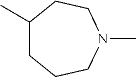 | 128 | NT | NT | NT | |
| R | H | H | OCH₃ | H | 1- | 0.25 | NT | NT | 64 | |
| | | | | | Adamantyl | | | | | |
| S | H | H | OH | H | 1- | 2 | NT | NT | 16 | |
| | | | | | Adamantyl | | | | | |
| T | H | CH₃ | CH₃ | H | c-Heptyl | >64 | NT | NT | NT | |
| U | H | CH₃ | CH₃ | H | c-Octyl | >64 | NT | NT | NT | |
| V | H | CH₃ | CH₃ | H | 1- | >64 | NT | NT | NT | |
| | | | | | Adamantyl | | | | | |
| W | H | CH₃ | CH₃ | H | 2- | >64 | NT | NT | NT | |
| | | | | | Adamantyl | | | | | |
| X | H | Cl | Cl | H | 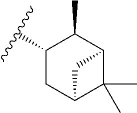 [SMILES: *[C@H]1C[C@H]2C[C@@H]([C@@H]1C)C2(C)C] | 0.0078 | NT | NT | NT | |
| Y | H | F | F | H | 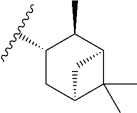 [SMILES: *[C@H]1C[C@H]2C[C@@H]([C@@H]1C)C2(C)C] | 0.0039 | NT | NT | NT | |
| Z | H | F | F | H | c-Octyl | 0.031 | NT | NT | NT | |
| AA | H | Cl | Cl | H | c-Octyl | 0.0039 | NT | NT | NT | |
| INH | | | | | | 0.04 | NT | NT | NT | |
| BB | H | H | Br | H | 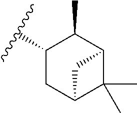 [SMILES: *[C@H]1C[C@H]2C[C@@H]([C@@H]1C)C2(C)C] | 0.0039- 0.0078 | NT | NT | NT | |

Positions 4 and 6 were further probed by replacing the dimethyl groups with halogen atoms (flourine and chlorine) to generate the di-fluoro- and dichloro-analogs 1x, 1y and 1z. The 4,6-dichloro-substituted analogs possess similar activity (1aa) or 2-fold lower activity (1x) in comparison to the 4,6-dimethyl analog (1h) while the 4,6-diflouro analog provided mixed results with compound 1y being as active as 1h and compound 1z displaying an 8-fold drop in activity.

### Results

Indoleamides are Active Against M. tuberculosis

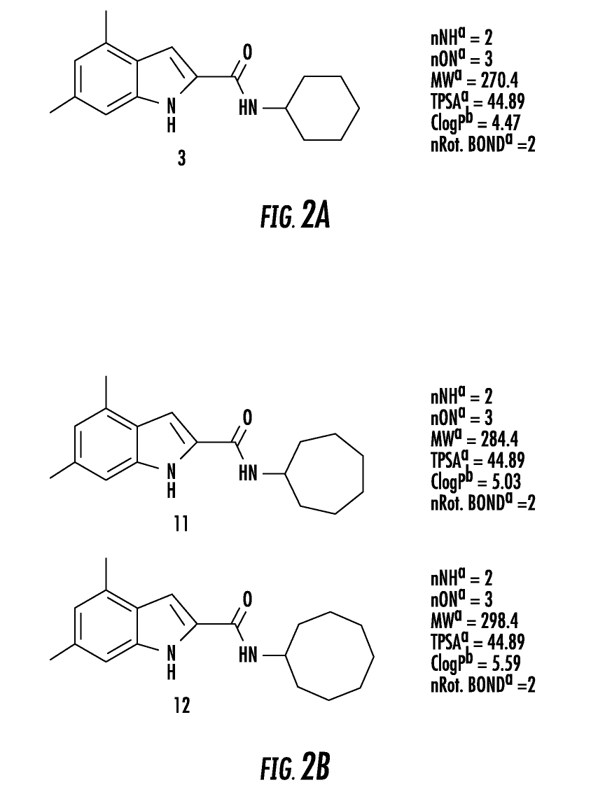

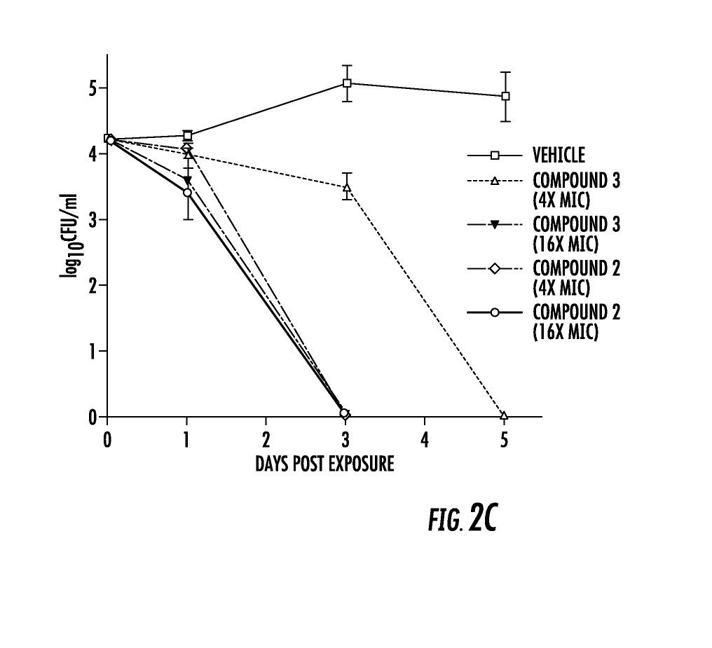

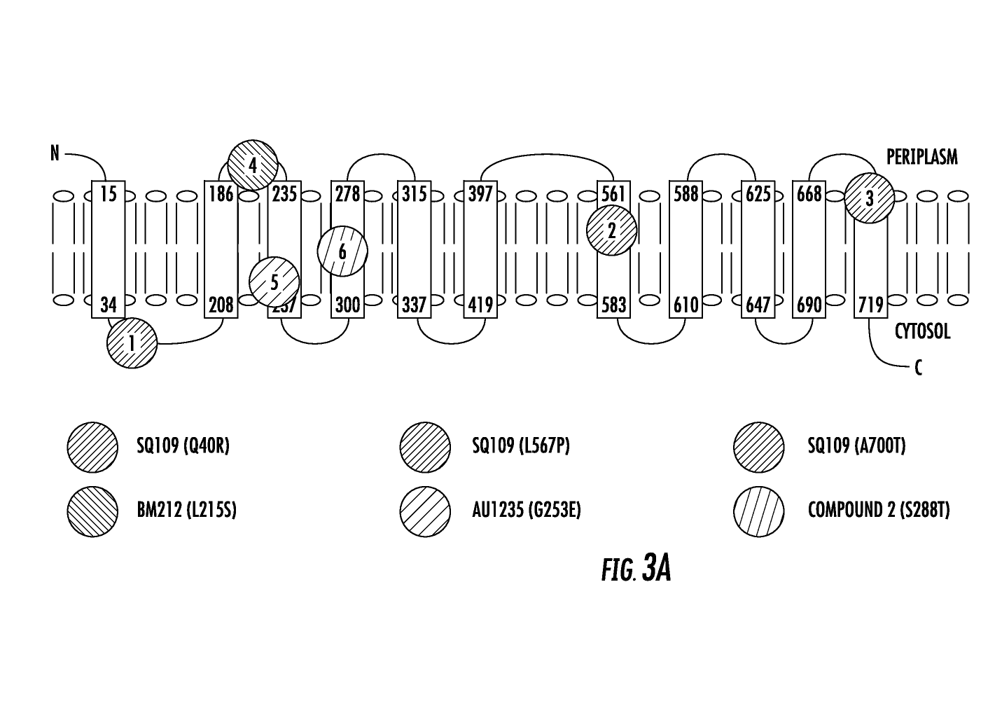

A high-throughput screen of compounds¹² identified a structurally simple indole-2-carboxamide, compound 3, with activity against M. tuberculosis (FIG. 2a). We used the indoleamide scaffold as a basis for the development of structural analogues, which yielded compounds 11 and 12 (FIG. 2b). The minimum inhibitory concentration (MIC) values of each of these compounds were determined against different M. tuberculosis strains, including a fully drug-susceptible laboratory reference strain, H37Rv, and five clinical isolates originally obtained from pulmonary TB patients in KwaZulu-Natal, South Africa^{11,15}. The patient isolates included a drug-susceptible strain (V4207), two confirmed MDR strains (V2475 and KZN494) and two XDR strains (TF274 and R506). As expected, the control strains H37Rv and V4207 were susceptible to the first-line and second-line drugs tested; the MIC values for compounds 3, 11 and 12 were 0.125-0.25, 0.0156-0.0313 and 0.0039 μg/mL, respectively²⁰, concentrations that are within a feasible range for translational utility. The MDR strains were resistant to isoniazid and rifampin but susceptible to the second-line drugs tested, and the XDR strains were resistant to all tested drugs¹¹. However, the indoleamide compounds exhibited MIC values of ≦1 μg/mL for all strains tested, suggesting that this structure class inhibits M. tuberculosis via a novel molecular interaction, and, importantly, that these compounds may be effective against MDR and XDR strains.

To further investigate the in vitro anti-mycobacterial activity of these indoleamide compounds, we determined their minimum bactericidal concentration (MBC) values against the H37Rv strain. For compounds 3, 11 and 12, the MBC values were 0.25, 0.0313 and 0.0078 μg/mL, respectively. Since compounds 11 and 12 exhibited lower MIC values for all M. tuberculosis strains tested than the original hit molecule, we assessed the kill kinetics of these two indoleamide derivatives at concentrations of 4× and 16× the MIC with the H37Rv reference strain. The 4× MIC of both compounds killed at least 4 log₁₀ₒ colony forming units (CFUs) within 3 or 5 days for compounds 11 and 12, respectively (FIG. 2c), suggesting aggressive bactericidal activity towards M. tuberculosis.

### Indoleamide Physicochemical Properties

In addition to their promising in vitro bactericidal activity against M. tuberculosis, the indoleamides have physicochemical properties that indicate great potential for absorption and permeation as orally available compounds. Namely, they comply with at least three of the four physicochemical parameters defined by the Lipinski “rule-of-five” which predict aqueous solubility and intestinal permeability²¹. All three indoleamide compounds had less than 5 hydrogen bond donors, less than 10 hydrogen bond acceptors, and molecular weights less than 500 g/mole (FIG. 2a,b). In terms of lipophilicity, compound 3 also had a CLogP value of less than 5, while compounds 11 and 12 had CLogP values just above 5. The ease of synthesis coupled with the promising physicochemical properties render these compounds attractive for further development as novel anti-tuberculosis drugs.

Furthermore, we assessed the potential cytotoxicity of our indoleamide compounds on mammalian cells using the Vero cell line. The half maximal inhibitory concentration (IC₅₀) value for Vero cell viability was high for all three tested compounds (>64 μg/mL for compounds 3 and 11, and 16 μg/mL for compound 3), indicating that they were non-toxic in this model system. Their low MIC values and toxicity profiles resulted in very high selectivity index values, ranging from >256 for compound 1 to >2048 for compound 11 and 4000 for compound 12.

We have demonstrated that compound 1y is bioavailable in vivo, as shown by the serum inhibition titration assay (SIT) (FIG. 6). Existence of the active form of 1y (N-(2,3,5 -methyl, 4-dimethyl)-4,6-difluoro-1H-indole-2-carboxamide) in mouse serum suggests reasonable PK/PD properties, thus further supporting the potential of this class of compounds as a novel anti-TB chemotype. Of particular note is the fact that 1y, although used at a higher dose, shows an activity comparable to that of isoniazid. Similar findings were made for compound BB (N-(2,3,5 -methyl, 4-dimethyl)-6-bromo-1H-indole-2-carboxamide).

mmpL3 Mutation Confers Resistance to Indoleamides

Initial in vitro experiments and structural analyses indicated that the indoleamides may represent a promising new anti-M. tuberculosis structure class for drug development; however, their bacterial target was unknown. Thus, we selected M. tuberculosis colonies with phenotypic resistance to compound 11 by growing the H37Rv reference strain on 7H10 agar plates containing a range of compound concentrations. We obtained one single CFU on a plate containing compound 11 at 8× the MIC. This isolate, referred to as IAR2 (indoleamide-resistant, compound 11) was able to multiply when inoculated into 7H9 liquid media with the same concentration of compound 11, indicating IAR2 was a true resistant mutant selected at a frequency of one in 3×10⁷ CFUs.

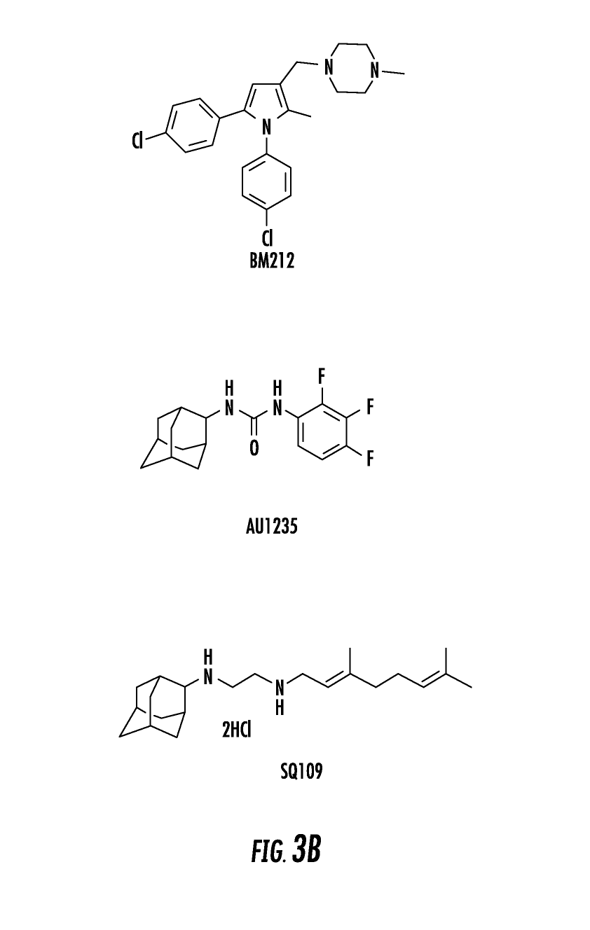

To identify mutations associated with resistance, whole genome sequencing was performed on both the IAR2 and parental H37Rv strains of M. tuberculosis using the Ion Torrent Personal Genome Machine platform. We obtained sequences for greater than 95% of each genome with approximately 30X coverage (Table 4), with the average read lengths of 98 and 118 bases for IAR2 and H37Rv, respectively. Relative to the H37Rv parental strain, the IAR2 genome contained a T to A single nucleotide polymorphism (SNP) at position 862 within the Rv0206c gene, encoding for MmpL3, a mycolic acid transporter. This SNP, which was further validated by Sanger sequencing, resulted in a serine to threonine missense mutation at position 288 of the cognate protein (FIG. 3a). This exact SNP was identified in 14/14 reads at this allele in the IAR2 genome (Table 5).

We then re-evaluated the MIC values of each of our indoleamide compounds for the IAR2 mutant and found the MIC to be much higher than the parental H37Rv strain (Table 6). The MIC upshift of this structure class ranged from 32 to 64-fold for compounds 11 and 12 to 1024-fold or greater for compound 3, suggesting that MmpL3, a mycolic acid transporter, is the target of the indoleamide compounds. Interestingly, in the last year, three different compounds have been reported to also target MmpL3: the urea derivative AU1235⁶, the pyrrole derivative BM212^{4,5}, and the diamine SQ109³ (FIG. 3b). We therefore determined the MIC values of these three compounds for the IAR2 mutant and found that the MIC for each compound was higher for IAR2 than for the parental H37Rv strain (Table 6).

### The IAR2 Mutant is not Cross-Resistant to TB Drugs

To assess the novelty of the microbial target of the indoleamide scaffold and the possible translational utility of this class of compounds for the treatment of both drug-susceptible and drug-resistant TB, we determined the MIC values of commonly used first-line (isoniazid, rifampin and ethambutol) and second-line (levofloxacin, moxifloxacin, kanamycin, capreomycin and amikacin) TB drugs on the IAR2 mutant and its H37Rv parental strain. All of the tested drugs exhibited the same MIC values for IAR2 as for H37Rv (except for rifampin, which actually had a lower MIC value for the mutant strain, Table 6). These results demonstrate that MmpL3 may be a validated molecular target in M. tuberculosis and that the S288T mutation in this target does not result in any cross-resistance to drugs currently used for TB treatment.

An Indoleamide Inhibits M. tuberculosis Growth In Vivo

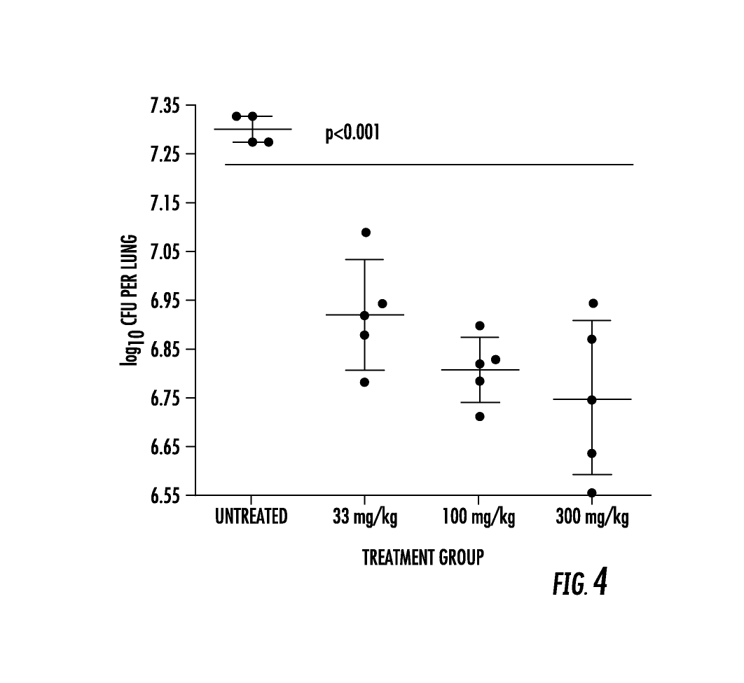

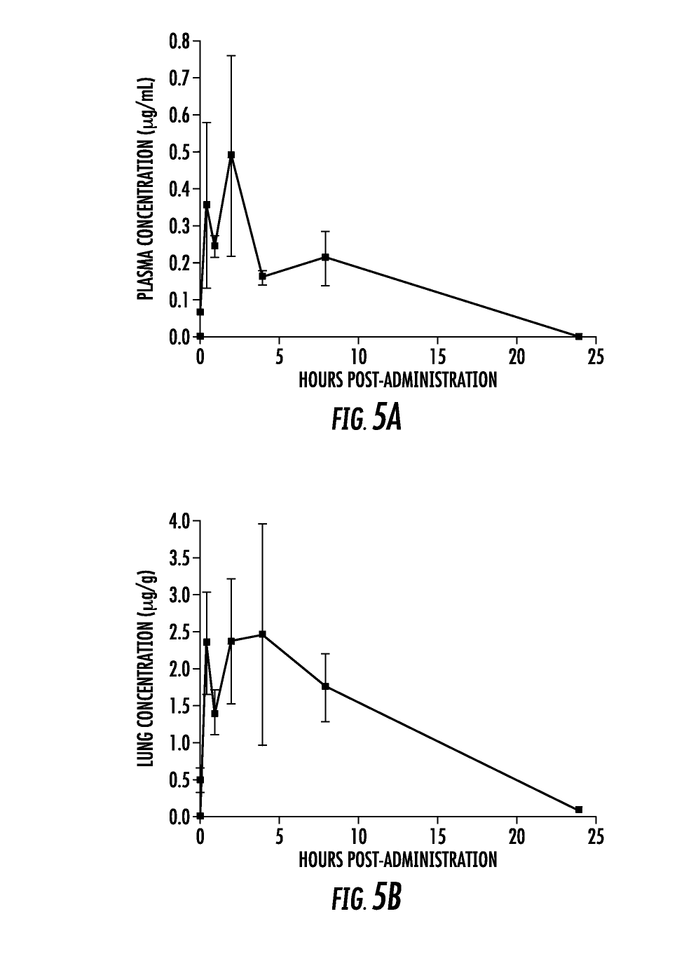

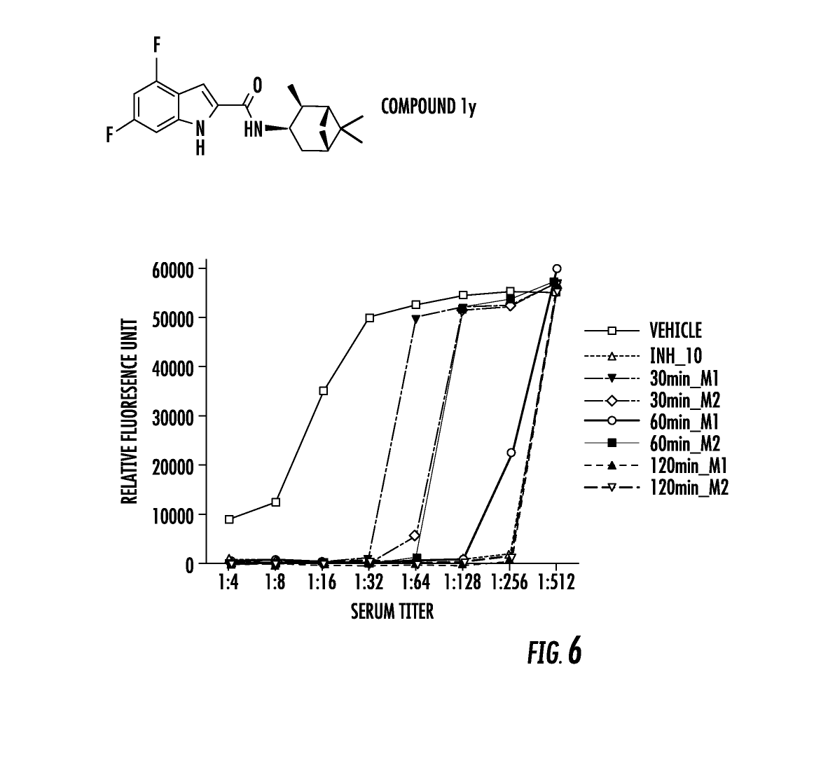

All of the in vitro experiments indicated that our indoleamide compounds may represent a new structure class active against a membrane transporter in M. tuberculosis (MmpL3) that is not targeted by existing TB drugs, prompting evaluation of the activity during in vivo infection. As compound 12 exhibited a dose-dependent mycobactericidal effect in vitro, we analyzed the effect of administration of this most potent compound to M. tuberculosis-infected mice. Female BALB/c mice were infected by aerosol with M. tuberculosis H37Rv (day 1 implantation of 3.0 log₁₀ CFU/lung), and two weeks after infection, when the bacterial burden was 6.5 logᵢₒ CFU/lung, compound 12 was administered daily to the mice by oral gavage at doses of 33, 100 and 300 mg/kg. After four weeks of treatment, the lung CFU counts were significantly lower in mice receiving any dose of compound 12 compared to untreated mice, and the bacterial burden in the lungs declined in a dose-dependent manner (FIG. 4, Table 7). Pharmacokinetic studies indicate that the 100 mg/kg dose results in a maximum concentration of 0.49 ug/mL in plasma and 2.47 ᵢug/g in the lungs (Table 8), well above the in vitro MIC value of 0.0039 ug/mL. Furthermore, in both plasma and lung, the concentration of compound 12 remained above the MIC for nearly 24 hours (FIG. 5). These data indicate that compound 12 is orally bioavailable in the mice and active against M. tuberculosis in vivo.

### Discussion

New drugs for the treatment of TB, including those that are effective against MDR- and XDR-TB, are greatly needed in the global effort to control this deadly disease. Whole-cell phenotypic screening has been demonstrated to be an effective method for the identification of novel structural classes of antimicrobial compounds, and in fact has proven more likely to generate lead compounds than rationale drug-design approaches'. However, appreciable limitations of this method include the lack of information regarding the target(s) of compounds, in vivo availability and tolerability. While the former limitation does not necessarily preclude the forward development of hit compounds, knowledge of the target(s) allows for effective lead optimization, providing a molecular basis for structure-activity relationship analyses and also indicating potential pathways for toxic activity within eukaryotic cells. The latter limitation is critical, and the demonstration of safe in vivo activity of a compound is absolutely essential for its continued development. Here, we describe a new structural class, the indoleamides, with promising activity against M. tuberculosis. Importantly, we have both identified the mycobacterial target and demonstrated in vivo availability and efficacy of this chemotype, overcoming two of the major hurdles in preclinical drug development.

Using the original hit compound 3 (FIG. 2a) identified from high-throughput screening, as well as two additional derivatives of this molecule (compounds 11 and 12, FIG. 2b), we demonstrated that these indoleamides were highly active against drug-susceptible, MDR and XDR M. tuberculosis strains²⁰, suggesting that these molecular entities may interact with a novel mycobacterial target. Indeed, the whole genome sequencing of an in vitro-selected mutant resistant to compound 11 revealed a mutation in the gene encoding for the mycolic acid transporter MmpL3 (FIG. 3a). Although currently not the known target of any licensed drug, MmpL3 has recently been identified as the target of several anti-mycobacterial compounds, strongly indicating that this transporter represents a bona fide target for anti-tuberculosis drug development. Our indoleamide-resistant mutant, IAR2, exhibited full sensitivity to currently used first- and second-line TB drugs (Table 3), indicating a lack of cross-resistance. Importantly, we also demonstrated that an indoleamide derivative (compound 12) was orally bioavailable and active against M. tuberculosis in a mouse model of TB (FIG. 4). These studies suggest that the indoleamide structural class represents a valuable source of possible agents effective against both drug-susceptible and drug-resistant TB. Interestingly, the indoleamide structural class was also identified to be active on M. tuberculosis by an independent group²², verifying the antitubercular property of this class.

The mycobacterial MmpL proteins belong to the resistance, nodulation and [cell] division (RIND) family of membrane transporters²³. RND family proteins are known to mediate the transport of a wide variety of substrates, including antimicrobial compounds, across cell membranes, and are also established as virulence factors for several bacterial pathogens'. M. tuberculosis strains encode up to 14 known MmpL family proteins, of which MmpL3 has been the least characterized due to difficulties in deleting its cognate gene, suggesting essentiality for the microorganism^{23,25,26}. Interestingly, MmpL3 has recently been identified as the target for a number of structurally distinct compounds: the pyrrole derivative BM212^{4,5}, the urea derivatives AU1235⁶ and 1-adamantyl-3-heteroaryl ureas²⁷, the diamine SQ109³ (FIG. 2b) and tetrahydropyrazolo[1,5-a]pyrimidine-3-carboxamide and N-benzyl-6′,7′-dihydrospiro[piperidine-4,4′-thieno[3,2-c]pyran] analogues²⁸; these studies have also revealed a role for MmpL3 in the transport of mycolic acids across the M. tuberculosis cell membrane. The molecular mechanisms involved in mycolic acid synthesis and assembly of the cell wall are well-appreciated molecular targets for both growth inhibition and killing of mycobacteria, being affected by key TB drugs including isoniazid and ethambutol²⁹. Thus, our finding that the indoleamide scaffold targets MmpL3 further corroborates the accumulating evidence that compound-based interactions with this protein interfere with M. tuberculosis growth. That we were able to target MmpL3 with an orally bioavailable compound suggests real translational possibility for the indoleamide structural class.

Our indoleamide-resistant M. tuberculosis strain, IAR2, was derived in vitro in the presence of compound 11, and we found that this strain contained a SNP in the gene encoding for MmpL3 resulting in an S288T amino acid change, which is predicted to occur in the fourth trans-membrane domain of the transporter (FIG. 3a). This alteration in MmpL3 was associated with decreased susceptibility to all of the indoleamides (compounds 3, 11 and 12), and interestingly also resulted in decreased susceptibility to the other known MmpL3-targeting compounds SQ109 and AU1235, and possibly BM212, as the increase in MIC value was only 2-fold (Table 6). In vitro-selected M. tuberculosis mutants resistant to these compounds were found to have different MmpL3-associated mutations, as illustrated in FIG. 3a. Thus, it is intriguing that the 5288T mutations conferred resistance to these compounds. However, it is possible that this amino acid substitution in the trans-membrane domain of MmpL3 alters the transporter structure in such a way that SQ109, BM212 and AU1235 cannot adequately access their targets within the protein. It would be of great interest to determine if the M. tuberculosis strains resistant to these compounds are also resistant to the indoleamides.

Certainly, our work provides further validation that MmpL3 is a viable target for anti-TB drug development. Furthermore, we demonstrated that the IAR2 mutant was fully susceptible to the commonly used first- and second-line TB drugs (Table 6). Considering that the AU1235-resistant mutant described by Grzegorzewicz and colleagues was also susceptible to the currently approved TB drugs⁶, our data strongly suggest that targeting MmpL3 is a valid strategy for the treatment of drug-resistant TB.

A key finding in our work is that the indoleamide structure class exhibited oral bioavailability and effectiveness in vivo in a mouse model of TB, thus demonstrating that these two large obstacles of high-throughput screening-based drug development can likely be overcome with members of this structure class. Moreover, lead optimization could result in increased in vivo activity of this group. The compound SQ109, which was identified from a phenotypic compound screen of a directed combinatorial library, has been shown to also be a very promising agent that also targets MmpL3, that was proven to be safe and well-tolerated in Phase I and early Phase II clinical trials^{30,31}. Our identification of an additional MmpL3-targeting class of compounds considerably bolsters the SQ109 work and could be developed in a complementary context, providing another effective, orally available option for TB treatment. Furthermore, it would be incredibly beneficial to examine whether combination of these two compounds could provide a synergistic effect for the complete inhibition of this essential target.

In summary, we have identified a novel structural class, the indoleamides, which interact with a validated target in M. tuberculosis, the MmpL3 transporter, and show vigorous activity against both drug-susceptible and drug-resistant (including MDR and XDR) M. tuberculosis strains. Our studies build upon and complement new and exciting findings in this field and strongly suggest that the indoleamides have serious translational potential for development into a real tool for TB treatment and control.

### REFERENCES

1. World Health Organization. Global Tuberculosis Report 2012. (2012).
2. United States Food and Drug Administration. Sirturo (bedaquiline) NDA 204384 Accelerated Approval Letter. Dec. 28, 2012. (2012).
3. Tahlan, K. et al. SQ109 targets MmpL3, a membrane transporter of trehalose monomycolate involved in mycolic acid donation to the cell wall core of Mycobacterium tuberculosis. Antimicrob. Agents Chemother. 56, 1797-1809 (2012).
4. La Rosa, V. et al. MmpL3 is the cellular target of the antitubercular pyrrole derivative BM212. Antimicrob. Agents Chemother. 56, 324-331 (2012).
5. Poce, G. et al. Improved BM212 MmpL3 inhibitor analogue shows efficacy in acute murine model of tuberculosis infection. PLoS One 8, e56980, DOI: 10.1371/journal.pone.0056980 (2013).
6. Grzegorzewicz, A. E. et al. Inhibition of mycolic acid transport across the Mycobacterium tuberculosis plasma membrane. Nat. Chem. Biol. 8, 334-341 (2012).
7. Kurdukar, R.; Subba Rao, N. V. Physiologically active compounds. VII. Synthesis of halo and nitro coumarones. Proc. Indian Acad. Sci. Section A 1963, 58, 336-342.
8. Dean, F. M.; Halewood, P.; Mongkolsuk, S.; Robertson, A.; Whalley, W. B. Usnic Acid. Part IX. A revised Structure for Usnolic Acid and the Resolution of Usnic Acid. J. Chem. Soc. 1953, 1250-1261.
9. Suzuki, T.; Horaguchi, T.; Shimizu, T.; Abe, T. Benzofuran Derivatives .1. On the Effects of Substituents in Benzofuran Syntheses. Bull. Chem. Soc. Jpn 1983, 56, 2762-2767.
10. Li, J. C.; Rush, T. S.; Li, W.; DeVincentis, D.; Du, X. M.; Hu, Y. H.; Thomason, J. R.; Xiang, J. S.; Skotnicki, J. S.; Tam, S.; Cunningham, K. M.; Chockalingam, P. S.; Morris, E. A.; Levin, J. I. Synthesis and SAR of highly selective MMP-13 inhibitors. Bioorg. Med. Chem.

Lett. 2005, /5, 4961-4966.
11. Pieroni, M.; Tipparaju, S. K.; Lun, S.; Song, Y.; Sturm, A. W.; Bishai, W. R.; Kozikowski, A. P. Pyrido[1,2-a]benzimidazole-Based Agents Active Against Tuberculosis (TB), Multidrug-Resistant (MDR) TB and Extensively Drug-Resistant (XDR) TB. Chem Med Chem 2011, 6, 334-342.
12. Collins, L. A.; Franzblau, S. G. Microplate Alamar blue assay versus BACTEC 460 system for high-throughput screening of compounds against Mycobacterium tuberculosis and Mycobacterium avium. Antimicrob. Agents Chemother. 1997, 41, 1004-1009.
13. Chopra, S.; Matsuyama, K.; Tran, T.; Malerich, J. P.; Wan, B.; Franzblau, S. G.; Lun, S.; Guo, H.; Maiga, M. C.; Bishai, W. R.; Madrid, P. B. Evaluation of gyrase B as a drug target in Mycobacterium tuberculosis. J. Antimicrob. Chemother. 2012, 67, 415-421.
14. Li, H. & Durbin, R. Fast and accurate short read alignment with Burrows-Wheeler transform. Bioinformatics 25, 1754-1760 (2009).
15. Ioerger, T. R.; Koo, S.; No, E. -G.; Chen, X.; Larsen, M. H.; Jacobs, W. R., Jr.; Pillay, M.; Sturm, A. W.; Sacchettini, J. C. Genome Analysis of Multi- and Extensively-Drug-Resistant Tuberculosis from KwaZulu-Natal, South Africa. Plos One 2009, 4, e7778.
16. Andries, K. et al. A diarylquinoline drug active on the ATP synthase of Mycobacterium tuberculosis. Science 307, 223-227 (2005).
17. Ananthan, S. et al. High-throughput screening for inhibitors of Mycobacterium tuberculosis H37Ry. Tuberculosis (Edinb) 89, 334-353 (2009).
18. Lilienkampf, A., Pieroni, M., Franzblau, S. G., Bishai, W. R. & Kozikowski, A. P. Derivatives of 3-isoxazolecarboxylic acid esters: a potent and selective compound class against replicating and nonreplicating Mycobacterium tuberculosis. Curr. Top. Med. Chem. 12, 729-734 (2012).
19. Mao, J., Wang, Y., Wan, B., Kozikowski, A. P. & Franzblau, S. G. Design, synthesis, and pharmacological evaluation of mefloquine-based ligands as novel antituberculosis agents. Chem Med Chem 2, 1624-1630 (2007).
20. Onajole, O. K. et al. Preliminary structure-activity relationships and biological evaluation of novel antitubercular indolecarboxamide derivatives against drug-susceptible and drug-resistant Mycobacterium tuberculosis strains. J. Med. Chem. 56, 4093-4103 (2013).
21. Payne, D. J., Gwynn, M. N., Holmes, D. J. & Pompliano, D. L. Drugs for bad bugs: confronting the challenges of antibacterial discovery. Nat. Rev. Drug Discov. 6, 29-40 (2007).
22. Ballell, L. et al. Fueling open-source drug discovery: 177 small-molecule leads against tuberculosis. Chem Med Chem 8, 313-321 (2013).
23. Domenech, P., Reed, M. B. & Barry, C. E., 3rd. Contribution of the Mycobacterium tuberculosis MmpL protein family to virulence and drug resistance. Infect. Immun. 73, 3492-3501 (2005).
24. Piddock, L. J. Multidrug-resistance efflux pumps—not just for resistance. Nat. Rev. Microbiol. 4, 629-636 (2006).
25. Tullius, M. V. et al. Discovery and characterization of a unique mycobacterial heme acquisition system. Proc. Natl. Acad. Sci. US. A. 108, 5051-5056 (2011).
26. Varela, C. et al. MmpL genes are associated with mycolic acid metabolism in mycobacteria and corynebacteria. Chem. Biol. 19, 498-506 (2012).
27. North, E. J. et al. Design, synthesis and anti-tuberculosis activity of 1-adamantyl-3-heteroaryl ureas with improved in vitro pharmacokinetic properties. Bioorg. Med. Chem. 21, 2587-2599 (2013).
28. Remunian, M. J. et al. Tetrahydropyrazolo[1,5-a]pyrimidine-3-carboxamide and N-benzyl-6′,7′-dihydrospiro[piperidine-4,4′-thieno[3,2-c]pyran] analogues with bactericidal efficacy against Mycobacterium tuberculosis targeting MmpL3. PLoS One 8, e60933, DOI: 10.1371/journal.pone.0060933 (2013).
29. Vilcheze, C. & Jacobs, W. R., Jr. The mechanism of isoniazid killing: clarity through the scope of genetics. Annu. Rev. Microbiol. 61, 35-50 (2007).
30. Sacksteder, K. A., Protopopova, M., Barry, C. E., 3rd, Andries, K. & Nacy, C. A. Discovery and development of SQ109: a new antitubercular drug with a novel mechanism of action. Future Microbiol. 7, 823-837 (2012).
31. Jia, L. et al. Pharmacodynamics and pharmacokinetics of SQ109, a new diamine-based antitubercular drug. Br. J. Pharmacol. 144, 80-87 (2005). Cole, S. T. et al. Deciphering the biology of Mycobacterium tuberculosis from the complete genome sequence. Nature 393, 537-544 (1998).

### Accession Codes

The genomic deep sequencing data have been deposited in the NCBI Trace and Short Read Archives (ncbi.nlm.nih.gov/Traces/home/) under accession code SRP030413.

1. A compound of formula I: 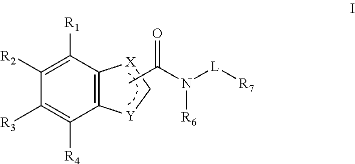
wherein
R₁, R₂, R₃ and R₄ are independently selected from H, alkyl, haloalkyl, alkoxy, halo and amino; X is CH, N or S; Y is O or NR₅; L is absent or C₁-C₄ alkyl; R₆ is H or alkyl; R₇ is C₃-C₁₂ cycloalkyl, C₃-C₁₂, C₅-C₈ heterocyclyl, C₆ aryl, C₅-C₆ heteroaryl or substituted or unsubstituted C₃-C₁₂ alkyl, or R₆ and R₇ together form a C₅-C₈ heterocyclyl; and Rs is H or alkyl, or a pharmaceutically acceptable salt, solvate or stereoisomer thereof.

2. A compound according to claim 1 of formula: 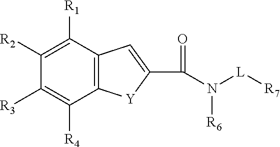
wherein L is absent or CH₂, or a pharmaceutically acceptable salt, solvate, or stereoisomer thereof.

3. A compound according to claim 2 wherein Y is NR₅, or a pharmaceutically acceptable salt, solvate, or stereoisomer thereof.

4. A compound according to claim 3 wherein R₂ and R₄ are H, or a pharmaceutically acceptable salt, solvate, or stereoisomer thereof.

5. A compound according to claim 4 wherein R₇ is C₈-C₁₂ cycloalkyl, or a pharmaceutically acceptable salt, solvate, or stereoisomer thereof.

6. A compound according to claim 5 wherein Ri and R3 are methyl or halogen, L is absent and R₅ is H, or a pharmaceutically acceptable salt, solvate, or stereoisomer thereof.

7. A compound according to claim 1 of formula 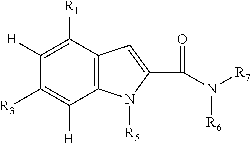
wherein R₁ and R₃ are Cl or F, and R₇ is C₆-C₁₂ cycloalkyl, or a pharmaceutically acceptable salt, solvate, or stereoisomer thereof.

8. A compound according to claim 7 wherein R₅ and R₆ are H, or a pharmaceutically acceptable salt, solvate, or stereoisomer thereof.

9. A compound according to claim 1 of formula I: 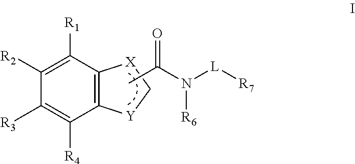
wherein
R₁, R₂, R₃ and R₄ are independently selected from H, alkyl, haloalkyl, alkoxy, halo and amino; X is CH, N or S; Y is O or NR₅; L is absent or C₁-C₄ alkyl; R₆ is H or alkyl;
R₇ is C₆-C₁₂ cycloalkyl, C₅-C₈ heterocyclyl or C₅-C₆ heteroaryl; and Rs is H or alkyl, or a pharmaceutically acceptable salt, solvate, or stereoisomer thereof.

10. A compound according to claim 9 wherein L is absent or CH₂, or a pharmaceutically acceptable salt, solvate, or stereoisomer thereof.

11. A compound according to claim 10 wherein Y is NR₅, or a pharmaceutically acceptable salt, solvate, or stereoisomer thereof.

12. A compound according to claim 11 wherein R₂ and R₄ are H, or a pharmaceutically acceptable salt, solvate, or stereoisomer thereof.

13. A compound according to claim 12 wherein R₁ and R₃ are methyl or halogen and L is absent, or a pharmaceutically acceptable salt, solvate, or stereoisomer thereof.

14. A compound according to claim 13 of formula 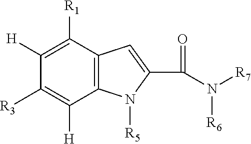
wherein R₁ and R₃ are Cl or F, or a pharmaceutically acceptable salt, solvate, or stereoisomer thereof.

15. A compound according to claim 14 wherein R₅ and R₆ are H, or a pharmaceutically acceptable salt, solvate, or stereoisomer thereof.

16. A compound according to claim 1 having the following formula: 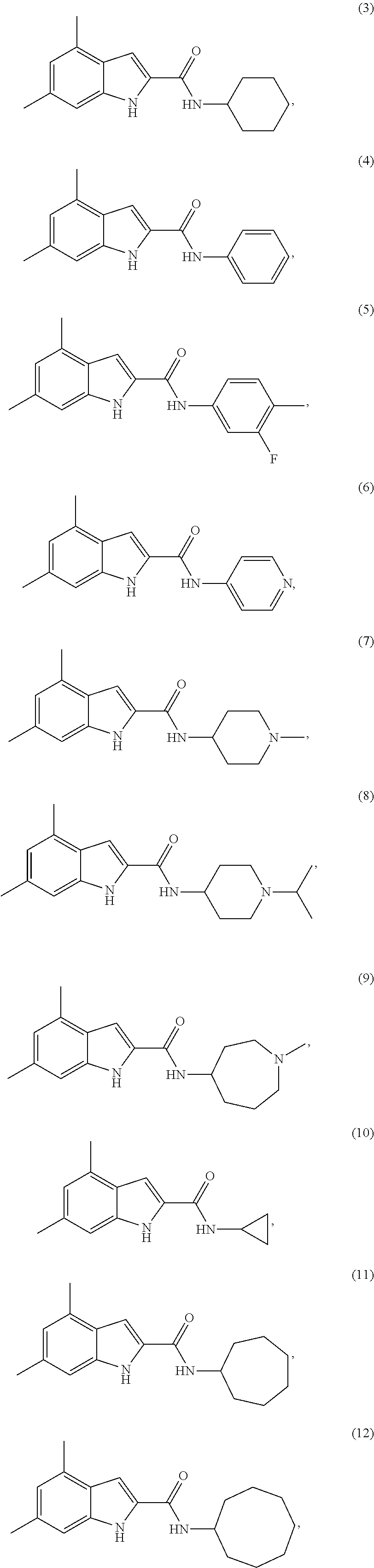 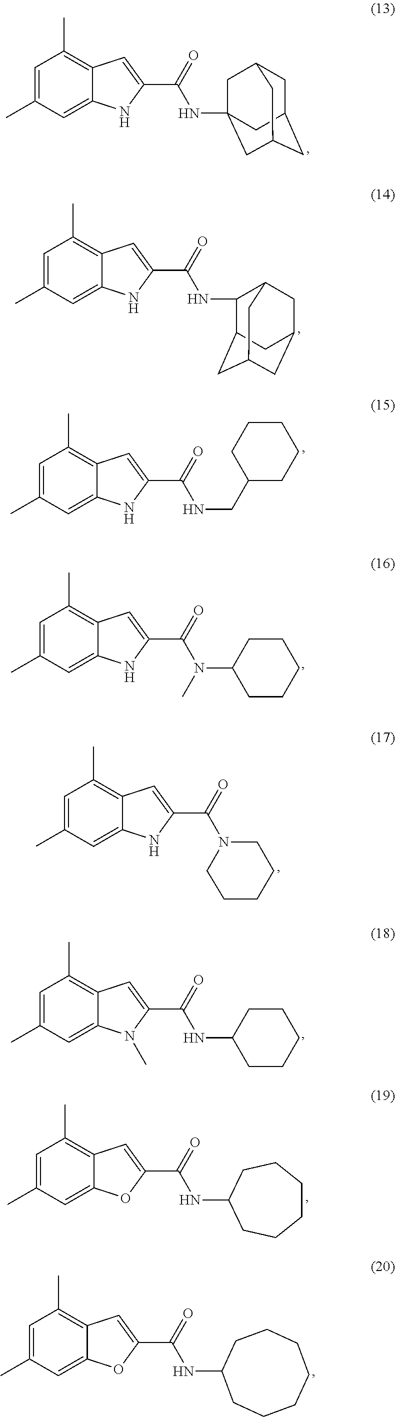 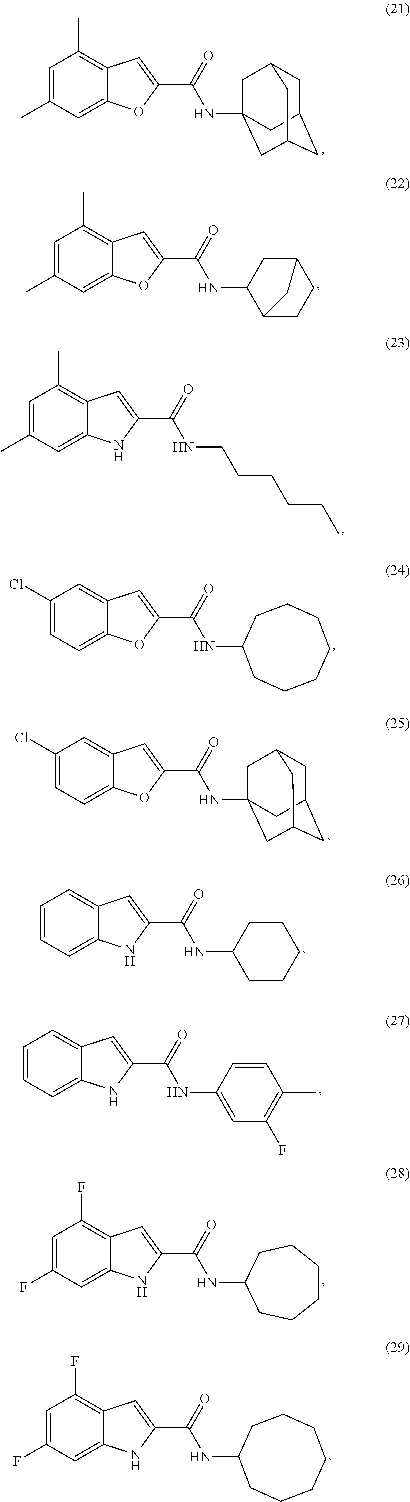 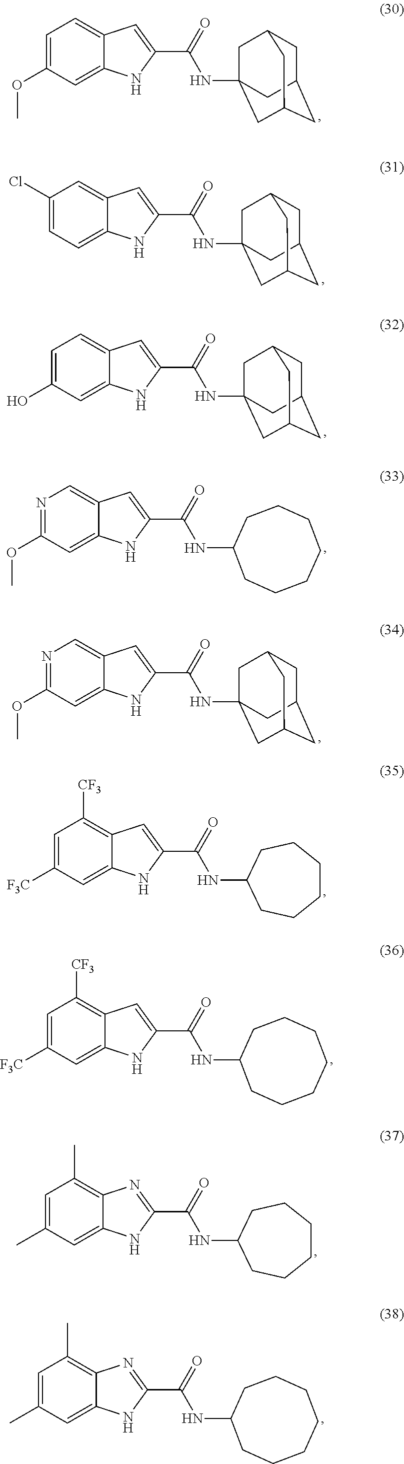 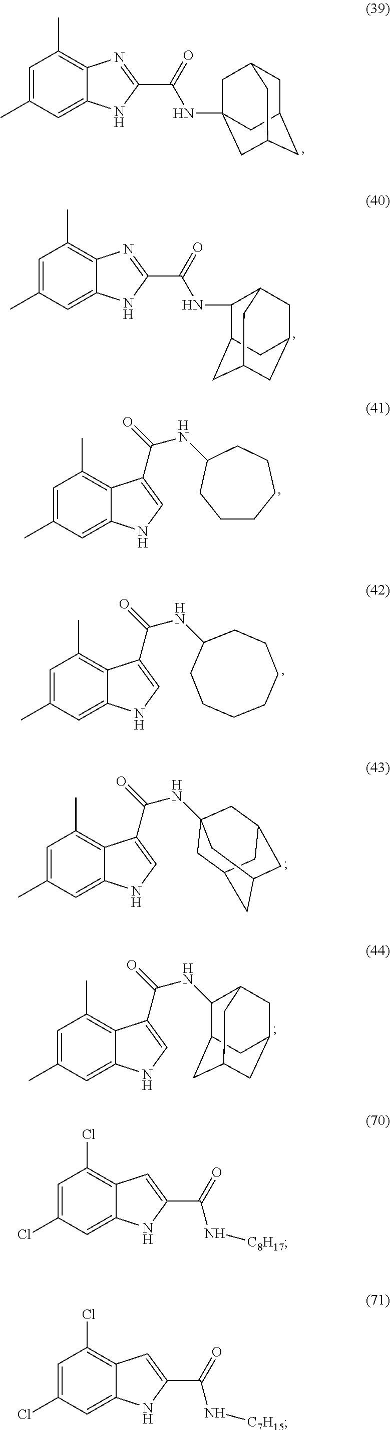 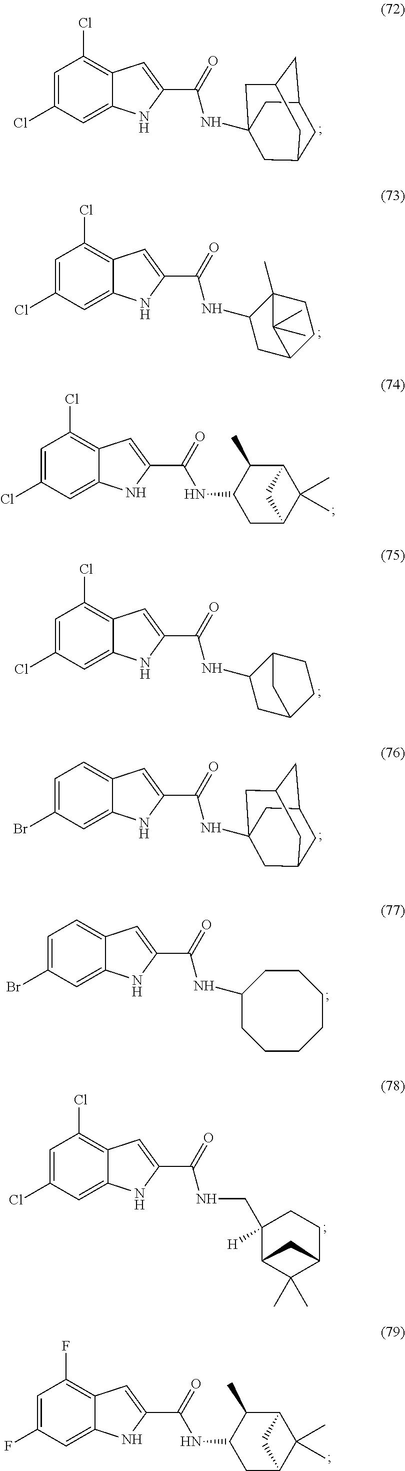 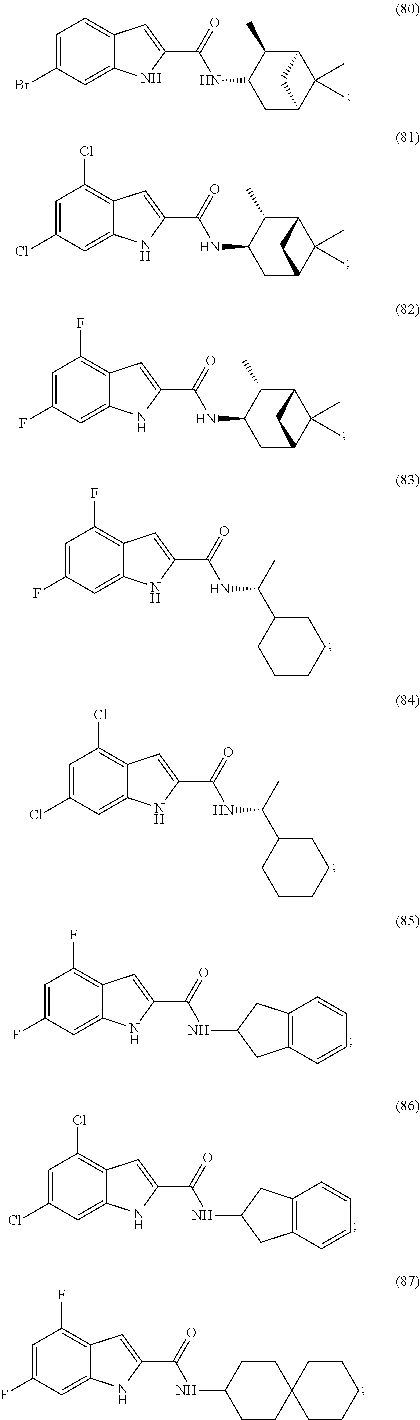 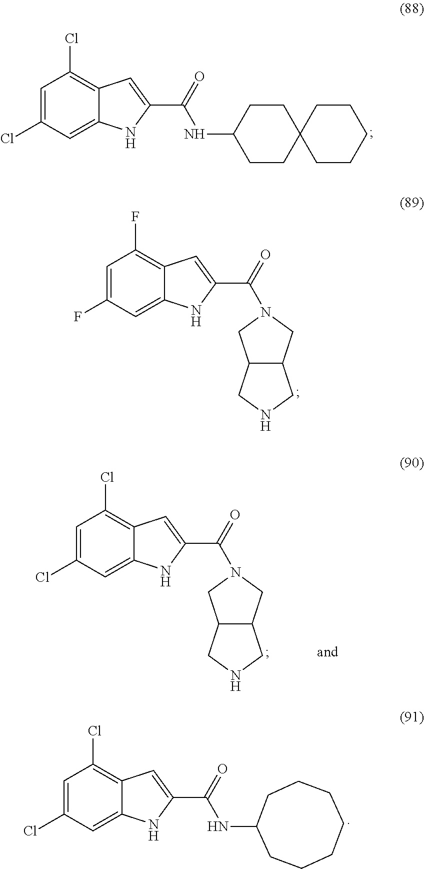 [SMILES: O=C(NC1CCC2(CCCCC2)CC1)c1cc2c(Cl)cc(Cl)cc2[nH]1 ; O=C(c1cc2c(F)cc(F)cc2[nH]1)N1CC2CNCC2C1 ; O=C(c1cc2c(Cl)cc(Cl)cc2[nH]1)N1CC2CNCC2C1 ; O=C(NC1CCCCCCC1)c1cc2c(Cl)cc(Cl)cc2[nH]1]
or a pharmaceutically acceptable salt, solvate, or stereoisomer thereof.

17. A compound according to claim 1 ehaving the following formula: 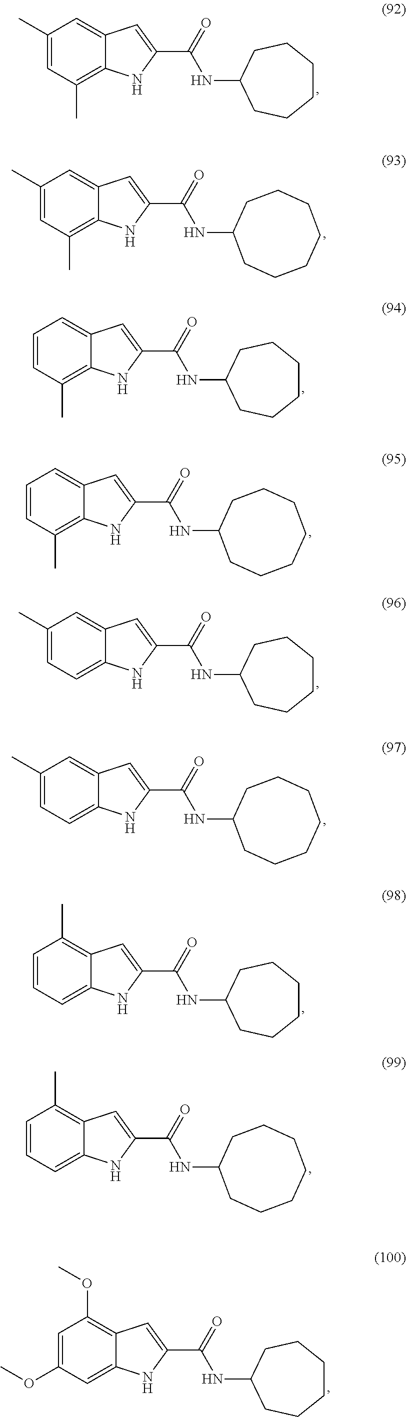 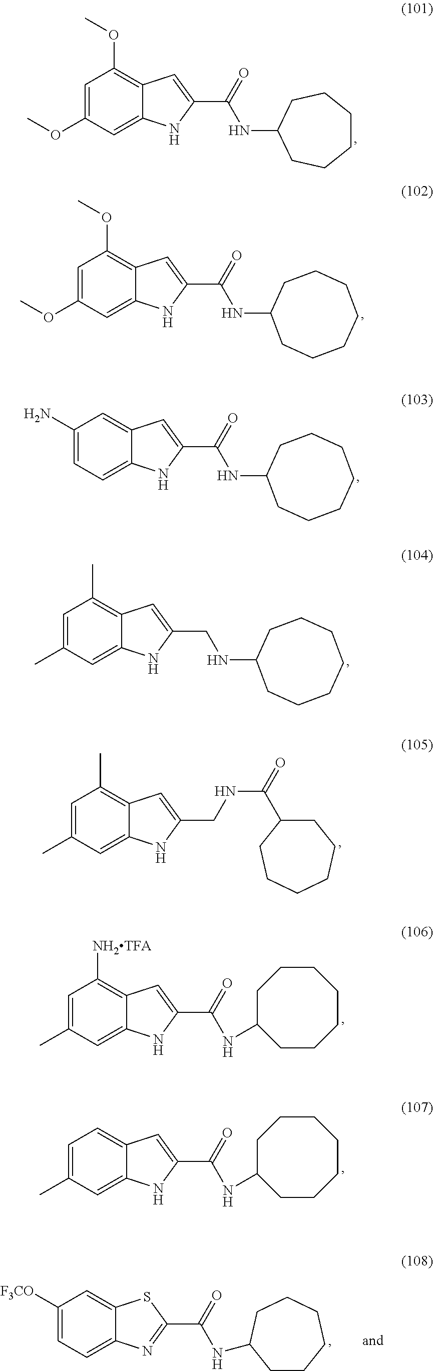 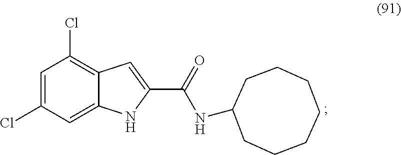 [SMILES: O=C(NC1CCCCCCC1)c1cc2c(Cl)cc(Cl)cc2[nH]1]
or a pharmaceutically acceptable salt, solvate, or stereoisomer thereof.

18. A compound according to claim 1 ef having the following formula 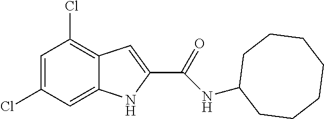 [SMILES: O=C(NC1CCCCCCC1)c1cc2c(Cl)cc(Cl)cc2[nH]1]
or a pharmaceutically acceptable salt, solvate, or stereoisomer thereof.

19. A pharmaceutical composition comprising one or more compounds according to claim 1, and a pharmaceutically acceptable carrier.

20. The pharmaceutical composition of claim 19, further comprising at least one or more biologically active agents.

21. The pharmaceutical composition of claim 19, wherein the at least one or more biologically active agents includes antimycotic agents such as isoniazid and rifampin.

22. A method for the treatment of tuberculosis in a subject in need thereof comprising administering an effective amount of one or more compounds of claim 1 to the subject.

23. A method for the treatment of tuberculosis in a subject in need thereof comprising administering an effective amount of a pharmaceutical composition comprising one or more compounds of claim 1, and at least one or more biologically active agents, and a pharmaceutically acceptable carrier, to the subject.

24. The method of claim 22, wherein the tuberculosis is MDR or XDR tuberculosis.

25. The method of claim 23, wherein the tuberculosis is MDR or XDR tuberculosis.
